(19) United States
(12) Patent Application Publication
(10) Pub. No.: US 2004/0068012 A1
(43) Pub. Date: Apr. 8, 2004

(54) Sulfonamides having antiangiogenic and anticancer activity

(76) Inventors: Kenneth M. Comess, Winnetka, IL (US); Scott A. Erickson, Zion, IL (US); Jack Henkin, Highland Park, IL (US); Douglas M. Kalvin, Buffalo Grove, IL (US); Megumi Kawai, Libertyville, IL (US); Ki Hwan Kim, Vista, CA (US); Nwe Y. BaMaung, Niles, IL (US); Chang Hoon Park, Libertyville, IL (US); George S. Sheppard, Wilmette, IL (US); Anil Vasudevan, Gurnee, IL (US); Jieyi Wang, Lake Bluff, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/267,081

(22) Filed: Oct. 8, 2002

(51) Int. Cl. A61K 31/195

(52) U.S. Cl. 514/562; 562/430

(57) ABSTRACT

Compounds having methionine aminopeptidase-2 inhibitory (MetAP2) are described. Also described are pharmaceutical compositions comprising the compounds, methods of treatment using the compounds, methods of inhibiting angiogenesis, and methods of treating cancer.

### TECHNICAL FIELD

The present invention relates to compounds having methionine aminopeptidase-2 inhibitory (MetAP2) activity useful for treating cancer and other conditions which arise from or are exacerbated by angiogenesis, pharmaceutical compositions comprising the compounds, methods of treatment using the compounds, methods of inhibiting angiogenesis, and methods of treating cancer.

### BACKGROUND OF THE INVENTION

Angiogenesis is the fundamental process by which new blood vessels are formed and is essential to a variety of normal body activities (such as reproduction, development, and wound repair). Although the process is not completely understood, it is believed to involve a complex interplay of molecules which both stimulate and inhibit the growth of endothelial cells, the primary cells of the capillary blood vessels. Under normal conditions these molecules appear to maintain the microvasculature in a quiescent state (i.e., one of no capillary growth) for prolonged periods that may last for weeks, or in some cases, decades. However, when necessary, such as during wound repair, these same cells can undergo rapid proliferation and turnover within as little as five days.

Although angiogenesis is a highly regulated process under normal conditions, many diseases (characterized as “angiogenic diseases”) are driven by persistent unregulated angiogenesis. Otherwise stated, unregulated angiogenesis may either cause a particular disease directly or exacerbate an existing pathological condition.

As the literature has established a causal link between inhibition of MetAP2 and the resultant inhibition of endothelial cell proliferation and angiogenesis (see Proc. Natl. Acad. Sci. USA 94: 6099-6103 (1997) and Chemistry and Biology, 4(6): 461-471 (1997)), it can be inferred that compounds which inhibit MetAP2 could serve as angiogenesis inhibitors.

### SUMMARY OF THE INVENTION

In its principle embodiment, the present invention provides a compound of formula (I)

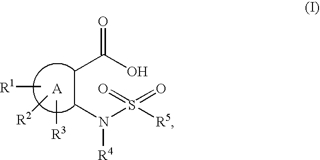

or a therapeutically acceptable salt thereof, wherein

A is a five- or six-membered aromatic or non-aromatic ring containing from zero to three atoms selected from the group consisting of nitrogen, oxygen, and sulfur; wherein the five-, or six-membered ring is optionally fused to a second five-, six-, or seven-membered aromatic or non-aromatic ring containing from zero to three atoms selected from the group consisting of nitrogen, oxygen, and sulfur;

R¹, R², and R³ are independently selected from the group consisting of hydrogen, alkoxy, alkoxyalkyl, alkyl, alkylsulfanyl, alkylsulfanylalkyl, amino, aminoalkyl, cycloalkyl, (cycloalkyl)alkyl, halo, haloalkoxy, haloalkyl, and hydroxyalkyl; provided that when A is phenyl, at least one of R¹, R², and R³ is other than hydrogen or C₁ alkyl;

R⁴ is selected from the group consisting of hydrogen, alkyl, alkylsulfanylalkyl, aryl, and arylalkyl; and

R⁵ is selected from the group consisting of alkyl, amino, aminoalkyl, aryl, arylalkenyl, arylalkyl, haloalkyl, heteroaryl, heteroarylalkenyl, heteroarylalkyl, and heterocycle.

In a preferred embodiment, the present invention provides a compound of formula (II)

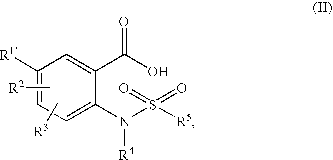

or a therapeutically acceptable salt thereof, wherein

R^{1′} is selected from the group consisting of alkoxy, alkoxyalkyl, C₂-C₁₀ alkyl, alkylsulfanyl, alkylsulfanylalkyl, amino, aminoalkyl, cycloalkyl, (cycloalkyl)alkyl, halo, haloalkoxy, and haloalkyl; and

R², R³, R⁴, and R⁵ are as defined for formula (I).

In another preferred embodiment, the present invention provides a compound of formula (III)

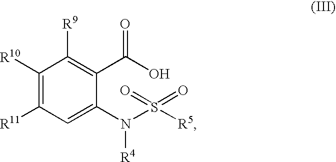

or a therapeutically acceptable salt thereof, wherein

R⁴ and R⁵ are as defined for formula (I); and

R⁹ is hydrogen and R¹⁰ and R¹¹, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl; or

R¹¹ is hydrogen and R⁹ and R¹⁰, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl.

In another preferred embodiment, the present invention provides a compound of formula (IV)

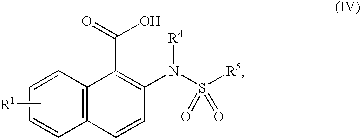

or a therapeutically acceptable salt thereof, wherein

R¹, R⁴, and R⁵ are as defined for formula (I).

In a more preferred embodiment, the present invention provides a compound of formula (IV), or a therapeutically acceptable salt thereof, wherein R⁵ is aryl. In another more preferred embodiment, the aryl is unsubstituted, monosubstituted, disubstituted, or trisubstituted.

In another more preferred embodiment, the present invention provides a compound of formula (IV), or a therapeutically acceptable salt thereof, wherein R⁵ is heteroaryl.

In another more preferred embodiment, the present invention provides a compound of formula (IV), or a therapeutically acceptable salt thereof, wherein R⁵ is selected from the group consisting of alkyl, arylalkenyl, arylalkyl, and haloalkyl.

In another embodiment, the present invention provides a method of inhibiting angiogenesis comprising administering to a patient in need of such treatment a therapeutically effective amount of a compound of formula (I)

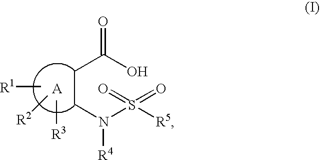

or a therapeutically acceptable salt thereof, wherein

A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur;

R¹, R², and R³ are independently selected from the group consisting of hydrogen, alkoxy, alkoxyalkyl, alkyl, alkylsulfanyl, alkylsulfanylalkyl, amino, aminoalkyl, cycloalkyl, (cycloalkyl)alkyl, halo, haloalkoxy, haloalkyl, and hydroxyalkyl;

R⁴ is selected from the group consisting of hydrogen, alkyl, alkylsulfanylalkyl, aryl, and arylalkyl; and

R⁵ is selected from the group consisting of alkyl, amino, aminoalkyl, aryl, arylalkenyl, arylalkyl, haloalkyl, heteroaryl, heteroarylalkenyl, heteroarylalkyl, and heterocycle.

In a preferred embodiment, the present invention provides a method of inhibiting angiogenesis comprising administering to a patient in need of such treatment a therapeutically effective amount of a compound of formula (V)

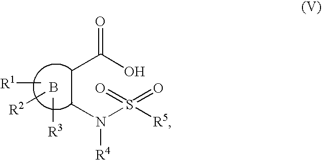

or a therapeutically acceptable salt thereof, wherein

B is a five- or six-membered carbocyclic aromatic or non-aromatic ring; and

R¹, R², R³, R⁴, and R⁵ are as defined for formula (I).

In a more preferred embodiment, the present invention provides a method of inhibiting angiogenesis comprising administering to a patient in need of such treatment a therapeutically effective amount of a compound of formula (II)

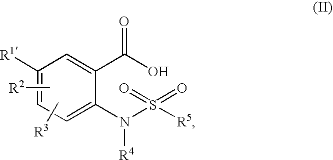

or a therapeutically acceptable salt thereof, wherein

R^{1′} is selected from the group consisting of alkoxy, alkoxyalkyl, alkyl, alkylsulfanyl, alkylsulfanylalkyl, amino, aminoalkyl, cycloalkyl, (cycloalkyl)alkyl, halo, haloalkoxy, and haloalkyl; and

R², R³, R⁴, and R⁵ are as defined for formula (I).

In another more preferred embodiment, the present invention provides a method of inhibiting angiogenesis comprising administering to a patient in need of such treatment a therapeutically effective amount of a compound of formula (II), or a therapeutically acceptable salt thereof, wherein R⁵ is aryl. In another more preferred embodiment, the aryl is unsubstituted, monosubstituted, or disubstituted.

In another more preferred embodiment, the present invention provides a method of inhibiting angiogenesis comprising administering to a patient in need of such treatment a therapeutically effective amount of a compound of formula (II), or a therapeutically acceptable salt thereof, wherein R⁵ is selected from the group consisting of alkyl and heteroaryl.

In another preferred embodiment the present invention provides a method of inhibiting angiogenesis comprising administering to a patient in need of such treatment a therapeutically effective amount of a compound of formula (VI)

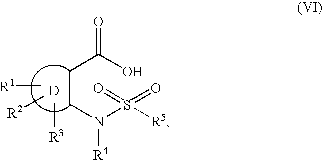

or a therapeutically acceptable salt thereof, wherein

D is a five- or six-membered aromatic or non-aromatic ring containing from zero to three atoms selected from the group consisting of nitrogen, oxygen, and sulfur; wherein the five- or six-membered ring is fused to a second five-, six-, or seven-membered aromatic or non-aromatic ring containing from zero to three atoms selected from the group consisting of nitrogen, oxygen, and sulfur; and

R¹, R², R³, R⁴, and R⁵ are as defined for formula (I).

In a more preferred embodiment, the present invention provides a method of inhibiting angiogenesis comprising administering to a patient in need of such treatment a therapeutically effective amount of a compound of formula (III)

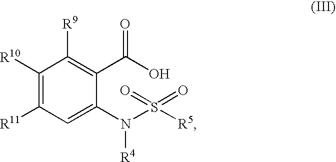

or a therapeutically acceptable salt thereof, wherein

R⁴ and R⁵ are as described in formula (I); and

R⁹ is hydrogen and R¹⁰ and R¹¹, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl; or

R¹¹ is hydrogen and R⁹ and R¹⁰, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl.

In another more preferred embodiment, the present invention provides a method of inhibiting angiogenesis comprising administering to a patient in need of such treatment a therapeutically effective amount of a compound of formula (III), or a therapeutically acceptable salt thereof, wherein R⁵ is aryl. In another more preferred embodiment, the aryl is unsubstituted, monosubstituted, disubstituted, or trisubstituted.

In another more preferred embodiment, the present invention provides a method of inhibiting angiogenesis comprising administering to a patient in need of such treatment a therapeutically effective amount of a compound of formula (III), or a therapeutically acceptable salt thereof, wherein R⁵ is heteroaryl.

In another more preferred embodiment, the present invention provides a method of inhibiting angiogenesis comprising administering to a patient in need of such treatment a therapeutically effective amount of a compound of formula (III), or a therapeutically acceptable salt thereof, wherein R⁵ is selected from the group consisting of alkyl, arylalkenyl, arylalkyl, and haloalkyl.

In another preferred embodiment, the present invention provides a method of inhibiting angiogenesis comprising administering to a patient in need of such treatment a therapeutically effective amount of a compound of formula (IV)

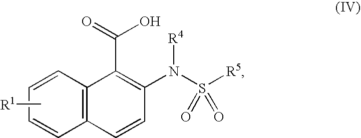

or a therapeutically acceptable salt thereof, wherein R¹, R⁴, and R⁵ are as defined for formula (I).

In another embodiment, the present invention provides a method of inhibiting methionine aminopeptidase-2 comprising administering to a patient in need of such treatment a therapeutically effective amount of a compound of formula (I), or a therapeutically acceptable salt thereof.

In another embodiment, the present invention provides a method of treating cancer comprising administering to a patient in need of such treatment a therapeutically effective amount of a compound of formula (I), or a therapeutically acceptable salt thereof.

In another embodiment, the present invention provides a method of treating cancer comprising administering to a patient in need of such treatment a therapeutically effective amount of a compound of formula (IV), or a therapeutically acceptable salt thereof.

In another embodiment, the present invention provides a pharmaceutical composition comprising a compound of formula (I), or a therapeutically acceptable salt thereof, in combination with a therapeutically acceptable carrier.

In another embodiment, the present invention provides a pharmaceutical composition comprising a compound of formula (IV), or a therapeutically acceptable salt thereof in combination with a therapeutically acceptable carrier.

### DETAILED DESCRIPTION OF THE INVENTION

As used in the present specification the following terms have the meanings indicated:

As used herein, the singular forms “a”, “an”, and “the” include plural reference unless the context clearly dictates otherwise.

The term “alkenyl,” as used herein, refers to a straight or branched chain group of two to ten carbon atoms containing at least one carbon-carbon double bond.

The term “alkoxy,” as used herein, refers to an alkyl group attached to the parent molecular moiety through an oxygen atom.

The term “alkoxyalkyl,” as used herein, refers to an alkoxy group attached to the parent molecular moiety through an alkyl group.

The term “alkoxycarbonyl,” as used herein, refers to an alkoxy group attached to the parent molecular moiety through a carbonyl group.

The term “alkyl,” as used herein, refers to a group of one to ten atoms derived from a straight or branched chain saturated hydrocarbon.

The term “C₁ alkyl,” as used herein, refers to an alkyl group with one carbon atom, i.e., a methyl group.

The term “C₂-C₁₀ alkyl,” as used herein, refers to an alkyl group two to ten carbon atoms in length.

The term “C₆-C₁₀ alkyl,” as used herein, refers to an alkyl group six to ten carbon atoms in length.

The term “alkylcarbonyl,” as used herein, refers to an alkyl group attached to the parent molecular moiety through a carbonyl group.

The term “alkylsulfanyl,” as used herein, refers to an alkyl group attached to the parent molecular moiety through a sulfur atom.

The term “alkylsulfanylalkyl,” as used herein, refers to an alkylsulfanyl group attached to the parent molecular moiety through an alkyl group.

The term “alkylsulfonyl,” as used herein, refers to an alkyl group attached to the parent molecular moiety through a sulfonyl group.

The term “armino,” as used herein, refers to —NR^{a}R^{b}, wherein R^{a} and R^{b} are independently selected from the group consisting of hydrogen, alkyl, alkylcarbonyl, cycloalkyl, (cycloalkyl)alkyl, and unsubstituted phenyl.

The term “aminoalkyl,” as used herein, refers to an amino group attached to the parent molecular moiety through an alkyl group.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a monocyclic cycloalkenyl group, as defined herein, a monocyclic cycloalkyl group, as defined herein, or another phenyl group. Tricyclic fused ring systems are exemplified by a bicyclic fused ring system fused to a monocyclic cycloalkenyl group, as defined herein, a monocyclic cycloalkyl group, as defined herein, or another phenyl group. Representative examples of aryl groups include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkenyl, alkoxy, alkoxycarbonyl, alkyl, alkylcarbonyl, alkylsulfonyl, aminoalkyl, a second aryl group, arylsulfonyl, carboxy, cyano, cyanoalkyl, halo, haloalkoxy, haloalkyl, heteroaryl, heterocycle, hydroxy, nitro, oxo, and —NR⁸R⁹, wherein R⁸ and R⁹ are independently selected from the group consisting of hydrogen, alkoxyalkyl, alkyl, alkylcarbonyl, aminoalkyl, cycloalkyl, (cycloalkyl)alkyl, and unsubstituted phenyl; and wherein the second aryl group, the aryl part of the arylsulfonyl, the heteroaryl, and the heterocycle can be optionally substituted with one, two, three, four, or five substituents independently selected from the group consisting of alkoxy, alkyl, cyano, halo, haloalkoxy, haloalkyl, and nitro.

The term “arylalkenyl,” as used herein, refers to an aryl group attached to the parent molecular moiety through an alkenyl group.

The term “arylalkyl,” as used herein, refers to an aryl group attached to the parent molecular moiety though an alkyl group.

The term “arylsulfonyl,” as used herein, refers to an aryl group attached to the parent molecular moiety through a sulfonyl group.

The term “carbonyl,” as used herein, refers to —C(O)—.

The term “carboxy,” as used herein, refers to —CO₂H.

The term “cyano,” as used herein, refers to —CN.

The term “cyanoalkyl,” as used herein, refers to a cyano group attached to the parent molecular moiety through an alkyl group.

The term “cycloalkenyl,” as used herein, refers to a non-aromatic cyclic or bicyclic ring system having three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Examples of cycloalkenyl groups include, but are not limited to, cyclohexenyl, octahydronaphthalenyl, and norbornylenyl.

The term “cycloalkyl,” as used herein, refers to a saturated monocyclic, bicyclic, or tricyclic hydrocarbon ring system having three to twelve carbon atoms. Examples of cycloalkyl groups include, but are not limited to, cyclopropyl, cyclopentyl, bicyclo[3.1.1]heptyl, and adamantyl.

The term “C₆-C₁₀ cycloalkyl,” as used herein, refers to a cycloalkyl group having six to ten carbon atoms.

The term “(cycloalkyl)alkyl,” as used herein, refers to a cycloalkyl group attached to the parent molecular moiety through an alkyl group.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes bicyclic systems where a heteroaryl ring is fused to a phenyl group, a monocyclic alkenyl group, as defined herein, a monocyclic cycloalkyl group, as defined herein, a heterocycle group, as defined herein, or an additional heteroaryl group; and tricyclic systems where a bicyclic system is fused to a phenyl group, a monocyclic cycloalkenyl group, as defined herein, a monocyclic cycloalkyl group, as defined herein, a heterocycle group, as defined herein, or an additional heteroaryl group. Examples of heteroaryl groups include, but are not limited to, benzothienyl, benzoxadiazolyl, cinnolinyl, dibenzofuranyl, furanyl, imidazolyl, indazolyl, indolyl, isoxazolyl, isoquinolinyl, isothiazolyl, naphthyridinyl, oxadiazolyl, oxadiazolyl, oxazolyl, thiazolyl, thienopyridinyl, thienyl, triazolyl, thiadiazolyl, pyridinyl, pyridazinyl, pyrimidinyl, pyrazinyl, pyrazolyl, pyrrolyl, quinolinyl, and triazinyl. The heteroaryl groups of the present invention can be optionally substituted with one, two, three, four, or five substituents independently selected from the group consisting of alkenyl, alkoxy, alkoxycarbonyl, alkyl, alkylcarbonyl, alkylsulfonyl, aminoalkyl, aryl, arylsulfonyl, carboxy, cyano, cyanoalkyl, halo, haloalkoxy, haloalkyl, a second heteroaryl group, heterocycle, hydroxy, nitro, oxo, and —NR⁸R⁹, wherein R⁸ and R⁹ are independently selected from the group consisting of hydrogen, alkoxyalkyl, alkyl, alkylcarbonyl, aminoalkyl, cycloalkyl, (cycloalkyl)alkyl, and unsubstituted phenyl; and wherein the aryl, the aryl part of the arylsulfonyl, the second heteroaryl group, and the heterocycle can be optionally substituted with one, two, three, four, or five substituents independently selected from the group consisting of alkoxy, alkyl, cyano, halo, haloalkoxy, haloalkyl, and nitro.

The term “heteroarylalkenyl,” as used herein, refers to a heteroaryl group attached to the parent molecular moiety through an alkenyl group.

The term “heteroarylalkyl,” as used herein, refers to a heteroaryl group attached to the parent molecular moiety through an alkyl group.

The term “heterocycle,” as used herein, refers to cyclic, non-aromatic, five-, six-, or seven-membered rings containing at least one atom selected from the group consisting of oxygen, nitrogen, and sulfur. The five-membered rings have zero or one double bonds and the six- and seven-membered rings have zero, one, or two double bonds. The heterocycle groups of the invention are connected to the parent molecular group through a substitutable carbon or nitrogen atom in the ring. The term “heterocycle” also includes bicyclic systems where a heterocycle ring is fused to a phenyl group, a monocyclic cycloalkenyl group, as defined herein, a monocyclic cycloalkyl group, as defined herein, or an additional monocyclic heterocycle group; and tricyclic systems where a bicyclic system is fused to a phenyl group, a monocyclic cycloalkenyl group, as defined herein, a monocyclic cycloalkyl group, as defined herein, or an additional monocyclic heterocycle group. Examples of heterocycles include, but are not limited to, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups of the present invention can be optionally substituted with one, two, three, four, or five substituents independently selected from the group consisting of alkoxy, alkoxycarbonyl, alkyl, alkylcarbonyl, cyano, halo, haloalkoxy, haloalkyl, and nitro.

The term “hydroxy,” as used herein, refers to —OH.

The term “hydroxyalkyl,” as used herein, refers to a hydroxy group attached to the parent molecular moiety through an alkyl group.

The term “nitro,” as used herein, refers to —NO₂.

The term “oxo,” as used herein, refers to ═O.

The term “sulfonyl,” as used herein, refers to —SO₂—.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

Because carbon-carbon double bonds exist in the present compounds, the invention contemplates various geometric isomers and mixtures thereof resulting from the arrangement of substituents around these carbon-carbon double bonds. It should be understood that the invention encompasses both isomeric forms, or mixtures thereof, which possess the ability to inhibit angiogenesis. These substituents are designated as being in the E or Z configuration wherein the term “E” represents higher order substituents on opposite sides of the carbon-carbon double bond, and the term “Z” represents higher order substituents on the same side of the carbon-carbon double bond.

In accordance with methods of treatment and pharmaceutical compositions of the invention, the compounds can be administered alone or in combination with other anticancer agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The antiangiogenic effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

Proc. Natl. Acad. Sci. USA 94: 6099-6103 (1997) and Chemistry and Biology, 4(6): 461-471 (1997) report that both AGM-1470 and ovalicin, a sequiterpene isolated from the fungus Pseudorotium ocalis have been found to bind to a common bifunctional protein, type 2-methionine aminopeptidase (MetAP-2) and conclude that MetAP2 plays a critical role in the proliferation of endothelial cells and may serve as a promising target for the development of new anti-angiogenic drugs.

Assays for the inhibition of catalytic activity of MetAP2 were performed in 96-well microtiter plates. Compounds to be tested (compounds of formula (1) where R² is hydrogen) were dissolved in dimethyl sulfoxide at 10 mM and diluted ten-fold in assay buffer (50 mM HEPES, pH 7.4, 125 mM NaCl). Ten microliters of solution of each compound to be tested for inhibition were introduced into each cell of the plate. Zero inhibition of enzyme activity was taken to be the result obtained in cells in which 10 μL of assay buffer was placed. A mixture totaling 90 μL per well and made up of 84 μL of assay buffer, 1 μL of L-amino acid oxidase (Sigma Catalog No. A-9378, ˜11 mg/mL), 1 μL of horseradish peroxidase (Sigma Catalog No. P-8451, dissolved in assay buffer at a concentration of 10 mg/mL), 1 μL of the tripeptide Met-Ala-Ser (Bachem) dissolved in assay buffer at concentration of 50 mM, 1 μL of ortho-dianisidine (Sigma Catalog No. D-1954, freshly made solution in water at a concentration of 10 mg/mL), and MetAP2 at a final concentration of 8 nM was rapidly mixed and added to each cell containing test or control compound. The absorbance at 450 nanometers was measured every 20 seconds over a period of twenty minutes using an automatic plate reader (Molecular Devices, CA, USA). The Vmax in mOD/min, calculated for each well, was used to represent MetAP2 activity. The IC₅₀ for each inhibitor was obtained by plotting the remaining activity versus inhibitor concentrations. Representative compounds of the present invention had IC₅₀'s between about 0.009 μM and >100 μM. Preferred compounds of the present invention had IC₅₀'s between about 0.009 μM and about 10 μM. Most preferred compounds had IC₅₀s, of between about 0.009 μM and about 0.1 μM.

As the literature has established a causal link between inhibition of MetAP2 and the resultant inhibition of endothelial cell proliferation and angiogenesis (see Proc. Natl. Acad. Sci. USA 94: 6099-6103 (1997) and Chemistry and Biology, 4(6): 461-471 (1997)), it can be inferred that the compounds of the invention, including, but not limited to those specified in the examples, possess antiangiogenic activity. As angiogenesis inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder, and urothelium), female genital tract (including cervix, uterus, and ovaries as well as choriocarcinoma and gestational trophoblastic disease), male genital tract (including prostate, seminal vesicles, testes, and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungicides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents. Additionally, the compounds of the invention can be used in the prevention of cancer (chemo prevention). The compounds of the invention can also be useful in the treatment of the aforementioned conditions by mechanisms other than the inhibition of angiogenesis.

Further uses include the treatment and prophylaxis of autoimmune diseases such as rheumatoid, immune and degenerative arthritis; psoriatic arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemagiomas, and capillary proliferation within atherosclerotic plaques; endometriosis; obesity; systemic sclerosis; juvenile angiofibroma; septic shock; cerebral edema (from head trauma); Osler-Webber Syndrome; myocardial angiogenesis; plaque neovascularization; telangiectasia; hemophiliac joints; angiofibroma; and wound granulation. Other uses include the treatment of diseases characterized by excessive or abnormal stimulation of endothelial cells, including not limited to intestinal adhesions, Crohn's disease, atherosclerosis, scleroderma, and hypertrophic scars, i.e., keloids. Another use is as a birth control agent, by inhibiting ovulation and establishment of the placenta. The compounds of the invention are also useful in the treatment of diseases that have angiogenesis as a pathologic consequence such as cat scratch disease (Rochele minutesalia quintosa) and ulcers (Helicobacter pylori). The compounds of the invention are also useful to reduce bleeding by administration prior to surgery, especially for the treatment of resectable tumors.

As MetAP2 inhibitors, the compounds of the invention also have use as antibacterial, antimalarial, and antileishmaniasis agents.

Synthetic Methods

Abbreviations which have been used in the descriptions of the scheme and the examples that follow are: DIAD for diisopropyl azodicarboxylate; DEAD for diethyl azodicarboxylate; TFA for trifluoracetic acid; dppf for 1,1′-bis(diphenylphosphino)ferrocene; DMSO for dimethylsulfoxide; THF for tetrahydrofuran; and DMF for N,N-dimethylformamide.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups A, R¹, R², R³, R⁴, and R⁵ are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

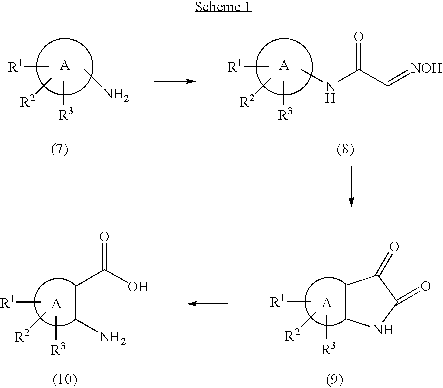

Scheme 1 shows the synthesis of compounds of formula (10). Compounds of formula (7) can be treated with chloral hydrate in the presence of a dehydrating agent, such as sodium sulfate, then treated with concentrated HCl and hydroxylamine hydrochloride to provide compounds of formula (8). Compounds of formula (8) can be treated with concentrated sulfuric acid to provide compounds of formula (9). Conversion of compounds of formula (9) to compounds of formula (10) can be accomplished by treatment with sodium hydroxide and hydrogen peroxide.

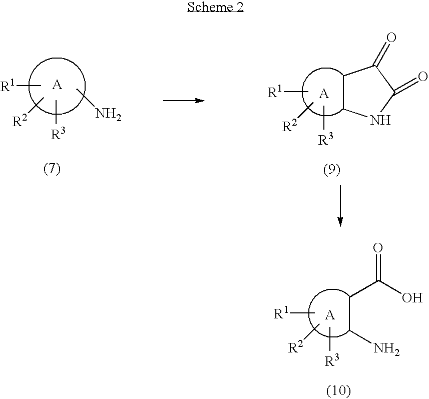

Scheme 2 shows an alternative preparation of compounds of formula (10). Compounds of formula (7) can be converted to compounds of formula (9) by treatment with glacial acetic acid and diethyl ketomalonate followed by treatment with potassium hydroxide. Conversion of compounds of formula (9) to compounds of formula (10) can be accomplished by the methods described in Scheme 1.

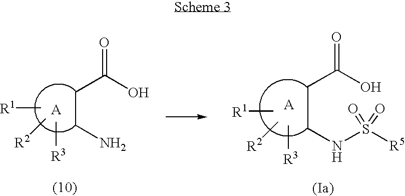

As shown in Scheme 3, compounds of formula (10) can be converted to compounds of formula (Ia) by treatment with chlorotrimethylsilane in the presence of a base such as triethylamine or pyridine, followed by sequential treatment with an appropriately substituted sulfonyl chloride (R⁵—SO₂Cl) and a strong acid such as HCl.

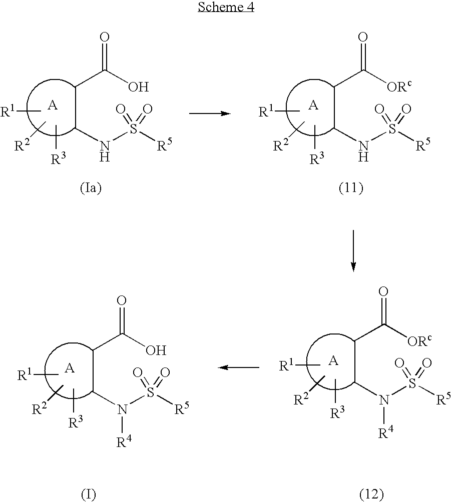

Scheme 4 shows the formation of compounds of formula (I) where R⁴ is other than hydrogen. Compounds of formula (Ia) (compounds of formula (I) where R4 is hydrogen) can be protected as an alkyl ester using conditions known to those of ordinary skill in the art to provide compounds of formula (11) (where R^{c} is alkyl). Compounds of formula (11) can be reacted with an appropriately substituted alcohol (R⁴—OH, where R⁴ is other than hydrogen) in the presence of a trialkyl- or triarylphosphine (such as tributylphosphine or triphenylphosphine) and either DIAD or DEAD to provide compounds of formula (12) where R⁴ is other than hydrogen. Hydrolysis of the ester using conditions known to those of ordinary skill in the art provides compounds of formula (I).

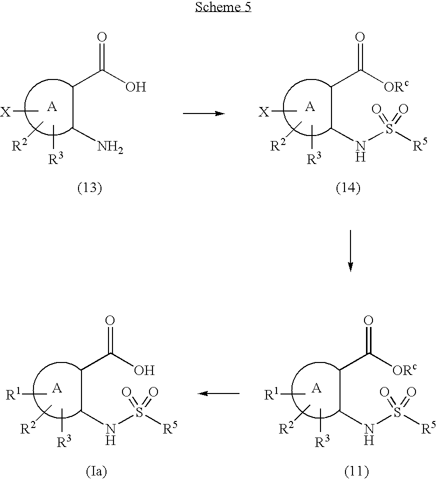

As shown in Scheme 5, compounds of formula (13) where X is Br, Cl, or I and R^{c} is an alkyl group (prepared by esterifying the corresponding carboxylic acid using methods known to those of ordinary skill in the art) can be converted to compounds of formula (Ia). Compounds of formula (13) can be converted to compounds of formula (14) by the methods described in Scheme 3. Compounds of formula (14) can be reacted with an appropriately substituted organometallic coupling partner (R¹-M, where M is a metal such as ZnCl or ZnBr) in the presence of a palladium catalyst (such as Pd(dppf)Cl₂) and copper iodide to provide compounds of formula (11). Hydrolysis of the ester with a hydroxide base such as sodium hydroxide or lithium hydroxide provides compounds of formula (Ia) (compounds of formula (I) where R⁴ is hydrogen).

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names which appeared to be consistent with ACD nomenclature.

### EXAMPLE 1

### 5-ethyl-2-[(phenylsulfonyl)amino]benzoic Acid

### EXAMPLE 1A

### N-(4-ethylphenyl)-2-(hydroxyimino)acetamide

A mixture of chloral hydrate (26.48 g, 160 mmol), anhydrous sodium sulfate (381 g, 2.68 mol), and 4-ethylaniline (18.6 mL, 150 mmol) in water (910 mL) at 80° C. was treated sequentially with concentrated HCl (20 mL) and a solution of hydroxylamine hydrochloride (31.8 g, 458 mmol) in water (150 mL). The mixture was heated to 80° C. for 1 hour, cooled to room temperature, and filtered. The filter cake was dried under vacuum to provide the desired product. MS (DCI) m/e 193 (M+H)⁺, 211 (M+NH₄)⁺.

### EXAMPLE 1B

### 5-ethyl-1H-indole-2,3-dione

Concentrated sulfuric acid (300 mL) at 50° C. was treated portionwise with Example 1A (28.8 g, 150 mmol), stirred at 50° C. for 30 minutes, poured over ice, stirred for 30 minutes, and filtered. The filter cake was dried under vacuum to provide the desired product. MS (DCI) m/e 176 (M+H)⁺, 193 (M+NH₄)⁺.

### EXAMPLE 1C

### 2-amino-5-ethylbenzoic Acid

A mixture of Example 1B (11.7 g, 66.9 mmol) in 1M NaOH (300 mL) was treated dropwise with 30% aqueous hydrogen peroxide (300 mL), heated to 50° C. for 30 minutes, cooled to room temperature, and filtered. The filtrate was adjusted to pH 4 with concentrated HCl, cooled to 4° C., and filtered. The filter cake was dried under vacuum to provide the desired product (4.46 g). MS (ESI(−)) m/e 164 (M−H)⁻.

### EXAMPLE 1D

### 5-ethyl-2-[(phenylsulfonyl)amino]benzoic Acid

A solution of Example 1C (0.033 g, 0.200 mmol) in dichloromethane (1 mL) was treated with 1M chlorotrimethylsilane in dichloromethane (440 μL, 0.044 mmol) and pyridine (56.6 μL, 0.70 mmol), shaken for 4 hours at ambient temperature, treated with a solution of benzenesulfonyl chloride (0.042 g, 0.24 mmol) in dichloromethane (1 mL), and shaken for 16 hours at ambient temperature. The mixture was concentrated, the residue was acidified to pH 1.0 with 5% aqueous HCl, and the solution was extracted with dichloromethane. The extracts were washed sequentially with water and brine, dried (MgSO₄), filtered, and concentrated. The concentrate was purified by C₁₈ reverse-phase HPLC with acetonitrile/water/0.5 mM ammonium acetate to provide the desired product. MS (ESI(+)) m/e 306 (M+H)⁺, 323 (M+NH₄)⁺, 328 (M+Na)⁺; (ESI(−)) m/e 304 (M−H)⁻; ¹H NMR (300 MHz, DMSO-d₆) δ 10.96 (br s, 1H), 7.78 (d, 2H), 7.73 (d, 1H), 7.64 (m, 1H), 7.55 (m, 2H), 7.42 (m, 2H), 2.54 (q, 2H), 1.10 (t, 3H).

### EXAMPLE 2

### 5-isopropyl-2-[(phenylsulfonyl)amino]benzoic Acid

### EXAMPLE 2A

### N-(2-bromo-4-isopropylphenyl)acetamide

A mixture of 2-bromo-4-isopropylaniline (5.05 g, 23.6 mmol), acetic anhydride (2.4 mL, 25 mmol), and triethylamine (3.5 mL, 25 mmol) in dichloromethane (25 mL) was stirred at ambient temperature for 4 days. The mixture was diluted with dichloromethane, washed sequentially with saturated aqueous Na₂CO₃ and 1M HCl, dried (MgSO₄), filtered, and concentrated to provide the desired product (5.85 g). MS (DCI) m/e 256, 258 (M+H)⁺; 273, 275 (M+NH₄)⁺.

### EXAMPLE 2B

### 2-(acetylamino)-5-isopropylbenzoic Acid

A mixture of Example 2A (3.33 g, 13.0 mmol), and [1,1′-bis(diphenylphosphino)ferrocene]dichloropalladium(II) dichloromethane complex (1.00 g, 1.2 mmol) in triethylamine (5.5 mL), dimethylformamide (25 mL), and water (5 mL) was shaken at 120° C. in a reactor pressurized with 850 psi of CO for 18 hours. The mixture was filtered, the filter cake was washed with ethyl acetate, and the combined filtrates were partitioned between diethyl ether and 1M NaOH. The aqueous phase was acidified with 12M HCl and extracted twice with ethyl acetate. The combined extracts were washed with brine, dried (MgSO₄), filtered, and concentrated to provide the desired product (2.38 g). MS (ESI(+)) m/e 222 (M+H)⁺, 244 (M+Na)⁺; (ESI(−)) m/e 220 (M−H)⁻.

### EXAMPLE 2C

### 2-amino-5-isopropylbenzoic Acid

A mixture of Example 2B (0.621 g, 2.81 mmol) and lithium hydroxide monohydrate (0.38 g, 9.0 mmol) in THF (6 mL) and water (6 mL) was stirred at 60° C. for 72 hours, acidified to pH 3.5 with 1M HCl, and extracted twice with ethyl acetate. The combined extracts were dried (MgSO₄), filtered, and concentrated. The concentrate was purified by C₁₈ reverse-phase HPLC with acetonitrile/water/0.1% TFA to provide the desired product. MS (ESI(+)) m/e 180 (M+H)⁺; (ESI(−)) m/e 178 (M−H)^{-.}

### EXAMPLE 2D

### 5-isopropyl-2-[(phenylsulfonyl)amino]benzoic Acid

The desired product was prepared by substituting Example 2C for Example 1C in Example 1D. MS (ESI(+)) m/e 320 (M+H)⁺, 337 (M+NH₄)⁺, 342 (M+Na)⁺; (ESI(−)) m/e 318 (M−H)⁻; ¹H NMR (300 MHz, DMSO-d₆) δ 10.98 (s, 1H), 7.80 (d, 2H), 7.73 (d, 1H), 7.64 (m, 1H), 7.58 (m, 2H), 7.43 (m, 2H), 2.84 (s, 1H), 1.14 (d, 6H).

### EXAMPLE 3

### 6-[(phenylsulfonyl)amino]-5-indanecarboxylic Acid

The desired product was prepared by substituting 5-indanamine for 4-ethylaniline in Examples 1A-D. MS (ESI(+)) m/e 318 (M+H)⁺, 335 (M+NH₄)⁺, 340 (M+Na)⁺; (ESI(−)) m/e 316 (M−H)⁻; ¹HNMR (300 MHz, DMSO-d₆) δ 11.18 (s, 1H), 7.79 (d, 2H), 7.72 (s, 1H), 7.62 (m, 1H), 7.55 (m, 2H), 7.41 (s, 1H), 2.85 (t, 2H), 2.78 (t, 2H), 1.97 (p, 2H).

### EXAMPLE 4

### 5-isobutyl-2-[(phenylsulfonyl)amino]benzoic Acid

### EXAMPLE 4A

### methyl 5-bromo-2-[(phenylsulfonyl)amino]benzoate

A mixture of methyl 2-amino-5-bromobenzoate (23.34 g, 101 mmol) in pyridine (100 mL) was treated with a solution of benzenesulfonyl chloride (14 mL, 110 mmol), stirred for 16 hours at ambient temperature, and concentrated. The concentrate was dissolved in dichloromethane, washed twice with 1N NaHSO₄, dried (MgSO₄), filtered, and concentrated. The concentrate was recrystallized from 3:1 ethanol/water (200 mL) to provide the desired product (33.4 g). MS (DCI) m/e 387, 389 (M+NH₄)⁺; ¹H NMR (300 MHz, DMSO-d₆) δ 10.32 (s, 1H), 7.91 (d, 1H), 7.80 (d, 1H), 7.77 (s, 1H), 7.75 (dd, 1H), 7.66 (d, 1H), 7.61-7.53 (m, 2H), 7.39 (d, 1H), 3.79 (s, 3H).

### EXAMPLE 4B

### 5-isobutyl-2-[(phenylsulfonyl)amino]benzoic Acid

A mixture of Example 4A (0.09 g, 0.24 mmol), Pd(dppf)Cl₂ (5 mol %), and CuI (6 mol %) was sealed using a crimper and treated with a solution of isobutylzinc bromide (0.5M in THF, 0.96 mL, 0.48 mmol). The reaction was heated in a single-mode microwave cavity in the Smith synthesizer at 160° C. for 600 seconds and filtered through a 1 micron PTFE syringe filter. The filtrate was concentrated, dissolved in 1:1 CH₃OH:DMSO (1.5 mL), and purified using a C₁₈ reverse-phase HPLC with acetonitrile/water/1% TFA. The purified ester was saponified by treatment with 10 equivalents of 2N NaOH in 1:1 CH₃OH:THF at 70° C. for 48 hours. The mixture was extracted with ethyl acetate and the extract was concentrated to provide the desired product. MS (ESI(+)) m/e 334 (M+H)⁺, 351 (M+NH₄)⁺, 356 (M+Na)⁺; ¹H NMR (300 MHz, DMSO-d₆) δ 10.90 (s, 1H), 7.80 (m, 2H), 7.40 (m, 3H), 7.10 (s, 1H), 6.80 (m, 1H), 6.50 (m, 1H), 2.20 (d, 2H), 1.70 (m, 1H), 0.80 (d, 6H).

### EXAMPLE 5

### 2-[(phenylsulfonyl)amino]-5-propylbenzoic Acid

The desired product was prepared by substituting propylzinc bromide for isobutylzinc bromide in Example 4B. MS (ESI(+)) m/e 320 (M+H)⁺, 337 (M+NH₄)⁺, 342 (M+Na)⁺; (ESI(−)) m/e 318 (M−H)⁻; ¹H NMR (300 MHz, DMSO-d₆) δ 10.90 (s, 1H), 7.78 (m, 2H), 7.38 (m, 3H), 7.20 (s, 1H), 6.80 (m, 1H), 6.60 (m, 1H), 2.20 (t, 2H), 1.50 (t, 2H), 0.90 (t, 3H).

### EXAMPLE 6

### 5-cyclopentyl-2-[(phenylsulfonyl)amino]benzoic Acid

The desired product was prepared by substituting cyclopentylzinc bromide for isobutylzinc bromide in Example 4B. MS (ESI(+)) m/e 346 (M+H)⁺, 363 (M+NH₄)⁺, 368 (M+Na)⁺; (ESI(−)) m/e 344 (M−H)⁻; ¹H NMR (300 MHz, DMSO-d₆) δ 10.92 (s, 1H), 7.80 (m, 2H), 7.40 (m, 3H), 7.20 (s, 1H), 6.80 (m, 1H), 6.60 (m, 1H), 2.60 (m, 1H), 1.82 (m, 2H), 1.62 (m, 2H), 1.50 (m, 2H), 1.40 (m, 2H).

### EXAMPLE 7

### 5-cyclohexyl-2-[(phenylsulfonyl)amino]benzoic Acid

The desired product was prepared by substituting cyclohexylzinc bromide for isobutylzinc bromide in Example 4B. MS (ESI(+)) m/e 360 (M+H)⁺, 377 (M+NH₄)⁺, 382 (M+Na)⁺; (ESI(−)) m/e 358 (M−H)⁻; ¹H NMR (300 MHz, DMSO-d₆) δ 10.94 (s, 1H), 7.80 (m, 2H), 7.40 (m, 3H), 7.20 (s, 1H), 6.80 (m, 1H), 6.60 (m, 1H), 2.25 (m, 1H), 1.6-1.75 (m, 5H), 1.20-1.35 (m, 5H).

### EXAMPLE 8

### 5-butyl-2-[(phenylsulfonyl)amino]benzoic Acid

The desired product was prepared by substituting butylzinc bromide for isobutylzinc bromide in Example 4B. MS (ESI(+)) m/e 334 (M+H)⁺, 351 (M+NH₄)⁺, 356 (M+Na)⁺; (ESI(−)) m/e 332 (M−H)⁻; ¹H NMR (300 MHz, DMSO-d₆) δ 10.94 (s, 1H), 7.80 (m, 2H), 7.40 (m, 3H), 7.20 (s, 1H), 6.80 (m, 1H), 6.60 (m, 1H), 2.24 (t, 2H), 1.40 (m, 2H), 1.20 (m, 2H), 0.91 (t, 3H).

### EXAMPLE 9

### 5-(3-methylbutyl)-2-[(phenylsulfonyl)amino]benzoic Acid

The desired product was prepared by substituting 3-methylbutylzinc bromide for isobutylzinc bromide in Example 4B. MS (ESI(+)) m/e 348 (M+H)⁺, 365 (M+NH₄)⁺, 370 (M+Na)⁺; (ESI(−)) m/e 346 (M−H)⁻; ¹H NMR (300 MHz, DMSO-d₆) δ 10.94 (s, 1H), 7.80 (m, 2H), 7.40 (m, 3H), 7.20 (s, 1H), 6.80 (m, 1H), 6.60 (m, 1H), 2.30 (t, 2H), 1.50 (m, 1H), 1.30 (m, 2H), 0.88 (d, 6H).

### EXAMPLE 10

### 5-(2-methylbutyl)-2-[(phenylsulfonyl)amino]benzoic Acid

The desired product was prepared by substituting 2-methylbutylzinc bromide for isobutylzinc bromide in Example 4B. MS (ESI(+)) m/e 348 (M+H)⁺, 365 (M+NH₄)⁺, 370 (M+Na)⁺; (ESI(−)) m/e 346 (M−H)⁻; ¹H NMR (300 MHz, DMSO-d₆) δ 10.92 (s, 1H), 7.80 (m, 2H), 7.40 (m, 3H), 7.20 (s, 1H), 6.80 (m, 1H), 6.50 (m, 1H), 2.30 (m, 1H), 2.10 (m, 1H), 1.45 (m, 1H), 1.30 (m, 1H), 1.05 (m, 1H), 0.85 (m, 3H), 0.75 (m, 3H).

### EXAMPLE 11

### 5-pentyl-2-[(phenylsulfonyl)amino]benzoic Acid

The desired product was prepared by substituting pentylzinc bromide for isobutylzinc bromide in Example 4B. MS (ESI(+)) m/e 348 (M+H)⁺, 365 (M+NH₄)⁺, 370 (M+Na)⁺; (ESI(−)) m/e 346 (M−H)⁻; ¹H NMR (300 MHz, DMSO-d₆) 6 (s, 1H), 7.80 (m, 2H), 7.40 (m, 3H), 7.20 (s, 1H), 6.80 (m, 1H), 6.50 (m, 1H), 2.30 (t, 2H), 1.42 (m, 2H), 1.22 (m, 4H), 0.89 (t, 3H).

### EXAMPLE 12

### 5-(2-ethylbutyl)-2-[(phenylsulfonyl)amino]benzoic Acid

The desired product was prepared by substituting 2-ethylbutylzinc bromide for isobutylzinc bromide in Example 4B. MS (ESI(+)) m/e 362 (M+H)⁺, 379 (M+NH₄)⁺, 384 (M+Na)⁺; (ESI(−)) m/e 360 (M−H)⁻; ¹H NMR (300 MHz, DMSO-d₆) δ 10.92 (s, 1H), 7.80 (m, 2H), 7.38 (m, 3H), 7.20 (s, 1H), 6.80 (m, 1H), 6.50 (m, 1H), 2.20 (d, 2H), 1.30 (m, 1H), 1.08 (m, 4H), 0.80 (t, 6H).

### EXAMPLE 13

### 5-hexyl-2-[(phenylsulfonyl)amino]benzoic Acid

The desired product was prepared by substituting hexylzinc bromide for isobutylzinc bromide in Example 4B. MS (ESI(+)) m/e 362 (M+H)⁺, 379 (M+NH₄)⁺, 384 (M+Na)⁺; (ESI(−)) m/e 360 (M−H)⁻; ¹H NMR (300 MHz, DMSO-d₆) δ 10.92 (s, 1H), 7.80 (m, 2H), 7.40 (m, 3H), 7.20 (s, 1H), 6.80 (m, 1H), 6.50 (m, 1H), 2.30 (t, 2H), 1.40 (m, 2H), 1.24 (m, 6H), 0.84 (t, 3H).

### EXAMPLE 14

### 2-{[(2-chloro-4-fluorophenyl)sulfonyl]amino}-5-ethylbenzoic Acid

The desired product was prepared by substituting 2-chloro-4-fluorobenzenesulfonyl chloride for benzenesulfonyl chloride in Example 1D. MS (ESI(+)) m/e 358 (M+H)⁺, 375 (M+NH₄)⁺, 380 (M+Na)⁺; (ESI(−)) m/e 356 (M−H)⁻; ¹H NMR (300 MHz, DMSO-d₆) δ 8.09 (dd, 1H), 7.66 (d, 1H), 7.47 (dd, 1H), 7.30 (td, 1H), 7.08 (d, 1H), 6.98 (dd, 1H), 2.44 (q, 2H), 1.08 (t, 3H).

### EXAMPLE 15

### 5-ethyl-2-{[(3-methylphenyl)sulfonyl]amino}benzoic Acid

The desired product was prepared by substituting 3-methylbenzenesulfonyl chloride for benzenesulfonyl chloride in Example 1D. MS (ESI(+)) m/e 320 (M+H)⁺, 337 (M+NH₄)⁺, 342 (M+Na)⁺; (ESI(−)) m/e 318 (M−H)⁻; ¹H NMR (300 MHz, DMSO-d₆) δ 7.65 (d, 1H), 7.55 (s, 1H), 7.51 (d, 1H), 7.33 (t, 1H), 7.29 (m, 1H), 7.23 (d, 1H), 7.00 (dd, 1H), 2.45 (q, 2H), 2.30 (s, 3H), 1.08 (t, 3H).

### EXAMPLE 16

### 5-ethyl-2-{[(2-fluorophenyl)sulfonyl]amino}benzoic Acid

The desired product was prepared by substituting 2-fluorobenzenesulfonyl chloride for benzenesulfonyl chloride in Example 1D. MS (ESI(+)) m/e 324 (M+H)⁺, 341 (M+NH₄)⁺, 346 (M+Na)⁺; (ESI(−)) m/e 322 (M−H)⁻; ¹H NMR (300 MHz, DMSO-d₆) δ 7.81 (td, 1H), 7.66 (d, 1H), 7.51 (m, 1H), 7.27-7.18 (m, 3H), 7.00 (dd, 1H), 2.45 (q, 2H), 1.08 (t, 3H).

### EXAMPLE 17

### 5-ethyl-2-{[(3-fluorophenyl)sulfonyl]amino}benzoic Acid

The desired product was prepared by substituting 3-fluorobenzenesulfonyl chloride for benzenesulfonyl chloride in Example 1D. MS (ESI(+)) m/e 324 (M+H)⁺, 341 (M+NH₄)⁺, 346 (M+Na)⁺; (ESI(−)) m/e 322 (M−H)⁻; ¹H NMR (300 MHz, DMSO-d₆) δ 7.65 (d, 1H), 7.55 (m, 1H), 7.50 (td, 1H), 7.44 (m, 1H), 7.32 (m, 1H), 7.24 (d, 1H), 7.04 (dd, 1H), 2.45 (q, 2H), 1.09 (t, 3H).

### EXAMPLE 18

### 5-ethyl-2-{[(4-fluorophenyl)sulfonyl]amino}benzoic Acid

The desired product was prepared by substituting 4-fluorobenzenesulfonyl chloride for benzenesulfonyl chloride in Example 1D. MS (ESI(+)) m/e 324 (M+H)⁺, 341 (M+N4)⁺, 346 (M+Na)⁺; (ESI(−)) m/e 322 (M−H)⁻; ¹H NMR (300 MHz, DMSO-d₆) δ 7.76 (dd, 2H), 7.65 (d, 1H), 7.28 (t, 2H), 7.24 (d, 1H), 7.05 (dd, 1H), 2.46 (q, 2H), 1.09 (t, 3H).

### EXAMPLE 19

### 2-{[(2-chlorophenyl)sulfonyl]amino}-5-ethylbenzoic Acid

The desired product was prepared by substituting 2-chlorobenzenesulfonyl chloride for benzenesulfonyl chloride in Example 1D. MS (ESI(+)) m/e 340, 342 (M+H)⁺, 357, 359 (M+NH₄)⁺, 362, 364 (M+Na)⁺; (ESI(−)) m/e 338, 340 (M−H)⁻; ¹H NMR (300 MHz, DMSO-d₆) δ 8.06 (d, 1H), 7.67 (d, 1H), 7.47 (m, 2H), 7.43 (m, 1H), 7.09 (d, 1H), 6.97 (dd, 1H), 2.44 (q, 2H), 1.08 (t, 3H).

### EXAMPLE 20

### 2-{[(3-chlorophenyl)sulfonyl]amino}-5-ethylbenzoic Acid

The desired product was prepared by substituting 3-chlorobenzenesulfonyl chloride for benzenesulfonyl chloride in Example 1D. MS (ESI(+)) m/e 340, 342 (M+H)⁺, 357, 359 (M+NH₄)⁺, 362, 364 (M+Na)⁺; (ESI(−)) m/e 338, 340 (M−H)⁻; ¹H NMR (300 MHz, DMSO-d₆) δ 7.65 (m, 3H), 7.53 (m, 1H), 7.48 (t, 1H), 7.22 (d, 1H), 7.04 (dd, 1H), 2.45 (q, 2H), 1.09 (t, 3H).

### EXAMPLE 21

### 2-{[(3,4-difluorophenyl)sulfonyl]amino}-5-ethylbenzoic Acid

The desired product was prepared by substituting 3,4-difluorobenzenesulfonyl chloride for benzenesulfonyl chloride in Example 1D. MS (ESI(+)) m/e 342 (M+H)⁺, 359 (M+NH₄)⁺, 364 (M+Na)⁺; (ESI(−)) m/e 340 (M−H)⁻; ¹H NMR (300 MHz, DMSO-d₆) 67.69 (m, 1H), 7.67 (d, 1H), 7.56 (m, 1H), 7.53 (m, 1H), 7.24 (d, 1H), 7.07 (dd, 1H), 2.47 (q, 2H), 1.10 (t, 3H).

### EXAMPLE 22

### 5-ethyl-2-[(1−naphthylsulfonyl)amino]benzoic Acid

The desired product was prepared by substituting 1-naphthalenesulfonyl chloride for benzenesulfonyl chloride in Example 1D. MS (ESI(+)) m/e 356 (M+H)⁺, 373 (M+NH₄)⁺, 378 (M+Na)⁺; (ESI(−)) m/e 354 (M−H)⁻; ¹H NMR (300 MHz, DMSO-d₆) δ 8.70 (d, 1H), 8.20 (d, 1H), 8.09 (d, 1H), 7.98 (d, 1H), 7.62 (t, 1H), 7.60-7.56 (m, 3H), 7.18 (d, 1H), 7.01 (d, 1H), 2.39 (q, 2H), 1.03 (t, 3H).

### EXAMPLE 23

### 5-ethyl-2-({[3-(trifluoromethyl)phenyl]sulfonyl}amino)benzoic Acid

The desired product was prepared by substituting 3-(trifluoromethyl)benzenesulfonyl chloride for benzenesulfonyl chloride in Example 1D. MS (ESI(+)) m/e 374 (M+H)⁺, 391 (M+NH₄)⁺, 396 (M+Na)⁺; (ESI(−)) m/e 372 (M−H)⁻; ¹H NMR (300 MHz, DMSO-d₆) δ 8.04 (d, 1H), 7.98 (s, 1H), 7.89 (d, 1H), 7.75 (t, 1H), 7.69 (d, 1H), 7.28 (d, 1H), 7.10 (dd, 1H), 2.49 (q, 2H), 1.13 (t, 3H).

### EXAMPLE 24

### 2-{[(2,3-dichlorophenyl)sulfonyl]amino}-5-ethylbenzoic Acid

The desired product was prepared by substituting 2,3-dichlorobenzenesulfonyl chloride for benzenesulfonyl chloride in Example 1D. MS (ESI(+)) m/e 374, 376 (M+H)⁺, 391, 393 (M+NH₄)⁺, 396, 398 (M+Na)⁺; (ESI(−)) m/e 372, 374 (M−H)⁻; ¹H NMR (300 MHz, DMSO-d₆) δ 8.12 (dd, 1H), 7.85 (dd, 1H), 7.73 (d, 1H), 7.53 (t, 1H), 7.22 (d, 1H), 7.18 (dd, 1H), 2.49 (q, 2H), 1.09 (t, 3H).

### EXAMPLE 25

### 2-{[(2,5-dichlorophenyl)sulfonYllamino}-5-ethylbenzoic Acid

The desired product was prepared by substituting 2,5-dichlorobenzenesulfonyl chloride for benzenesulfonyl chloride in Example 1D. MS (ESI(+)) m/e 374, 376 (M+H)⁺, 391, 393 (M+NH₄)⁺, 396, 398 (M+Na)⁺; (ESI(−)) m/e 372, 374 (M−H)⁻; ¹H NMR (300 MHz, DMSO-d₆) δ 7.99 (d, 1H), 7.67 (d, 1H), 7.54 (dd, 1H), 7.50 (d, 1H), 7.11 (d, 1H), 7.04 (dd, 1H), 2.45 (q, 2H), 1.09 (t, 3H).

### EXAMPLE 26

### 2-{[(3,5-dichlorophenyl)sulfonyl]amino}-5-ethylbenzoic Acid

The desired product was prepared by substituting 3,5-dichlorobenzenesulfonyl chloride for benzenesulfonyl chloride in Example 1D. MS (ESI(+)) m/e 374, 376 (M+H)⁺, 391, 393 (M+NH₄)⁺, 396, 398 (M+Na)⁺; (ESI(−)) m/e 372, 374 (M−H)⁻; ¹H NMR (300 MHz, DMSO-d₆) δ 7.84 (t, 1H), 7.70 (d, 1H), 7.67 (d, 2H), 7.30 (d, 1H), 7.26 (dd, 1H), 2.52 (q, 2H), 1.12 (t, 3H).

### EXAMPLE 27

### 2-{[(2-bromophenyl)sulfonyl]amino}-5-ethylbenzoic Acid

The desired product was prepared by substituting 2-bromobenzenesulfonyl chloride for benzenesulfonyl chloride in Example 1D. MS (ESI(+)) m/e 384, 386 (M+H)⁺, 401, 403 (M+NH₄)⁺, 406, 408 (M+Na)⁺; (ESI(−)) m/e 382, 384 (M−H)⁻; ¹H NMR (300 MHz, DMSO-d₆) δ 8.09 (dd, 1H), 7.69-7.66 (m, 2H), 7.49 (t, 1H), 7.37 (td, 1H), 7.06 (d, 1H), 6.96 (dd, 1H), 2.44 (q, 2H), 1.08 (t, 3H).

### EXAMPLE 28

### 2-{[(3-bromophenyl)sulfonyl]amino}-5-ethylbenzoic Acid

The desired product was prepared by substituting 3-bromobenzenesulfonyl chloride for benzenesulfonyl chloride in Example 1D. MS (ESI(+)) m/e 384, 386 (M+H)⁺, 401, 403 (M+NH₄)⁺, 406, 408 (M+Na)⁺; (ESI(−)) m/e 382, 384 (M−H)⁻; ¹H NMR (300 MHz, DMSO-d₆) δ 7.82 (t, 1H), 7.74-7.70 (m, 2H), 7.68 (d, 1H), 7.44 (t, 1H), 7.27 (d, 1H), 7.12 (dd, 1H), 2.48 (q, 2H), 1.10 (t, 3H).

### EXAMPLE 29

### 5-ethyl-2-{[(4-methylphenyl)sulfonyl]amino}benzoic Acid

The desired product was prepared by substituting 4-methylbenzenesulfonyl chloride for benzenesulfonyl chloride in Example 1D. MS (ESI(+)) m/e 320 (M+H)⁺, 337 (M+NH₄)⁺, 342 (M+Na)⁺; (ESI(−)) m/e 318 (M−H)⁻; ¹H NMR (300 MHz, DMSO-d₆) δ 7.65 (d, 1H), 7.60 (d, 2H), 7.26-7.22 (m, 3H), 7.00 (dd, 1H), 2.44 (q, 2H), 2.29 (s, 3H), 1.08 (t, 3H).

### EXAMPLE 30

### 2-{[(3-cyanophenyl)sulfonyl]amino}-5-ethylbenzoic Acid

The desired product was prepared by substituting 3-cyanobenzenesulfonyl chloride for benzenesulfonyl chloride in Example 1D. MS (ESI(+)) m/e 348 (M+NH₄)⁺, 353 (M+Na)⁺; (ESI(−)) m/e 329 (M−H)⁻; ¹H NMR (300 MHz, DMSO-d₆) δ 8.06 (s, 1H), 8.00 (d, 1H), 7.94 (d, 1H), 7.68 (d, 1H), 7.66 (m, 1H), 7.23 (d, 1H), 7.06 (dd, 1H), 2.44 (q, 2H), 1.09 (t, 3H).

### EXAMPLE 31

### 2-{[(4-cyanophenyl)sulfonyl]amino}-5-ethylbenzoic Acid

The desired product was prepared by substituting 4-cyanobenzenesulfonyl chloride for benzenesulfonyl chloride in Example 1D. MS (ESI(+)) m/e 348 (M+NH₄)⁺, 353 (M+Na)⁺; (ESI(−)) m/e 329 (M−H)⁻; ¹H NMR (300 MHz, DMSO-d₆) δ 7.92 (d, 2H), 7.86 (d, 2H), 7.65 (d, 1H), 7.22 (d, 1H), 7.05 (dd, 1H), 2.46 (q, 2H), 1.09 (t, 3H).

### EXAMPLE 32

### 2-{[(2,5-dimethylphenyl)sulfonyl]amino}-5-ethylbenzoic Acid

The desired product was prepared by substituting 2,5-dimethylbenzenesulfonyl chloride for benzenesulfonyl chloride in Example 1D. MS (ESI(+)) m/e 334 (M+H)⁺, 351 (M+NH₄)⁺, 356 (M+Na)⁺; (ESI(−)) m/e 332 (M−H)⁻; ¹H NMR (300 MHz, DMSO-d₆) δ 7.74 (s, 1H), 7.65 (d, 1H), 7.18 (d, 1H), 7.13 (d, 1H), 7.10 (d, 1H), 6.96 (dd, 1H), 2.49 (s, 3H), 2.43 (q, 2H), 2.29 (s, 3H), 1.08 (t, 3H).

### EXAMPLE 33

### 5-ethyl-2-{[(3-methoxyphenyl)sulfonyl]amino]benzoic Acid

The desired product was prepared by substituting 3-methoxybenzenesulfonyl chloride for benzenesulfonyl chloride in Example 1D. MS (ESI(+)) m/e 336 (M+H)⁺, 353 (M+NH₄)⁺, 358 (M+Na)⁺; (ESI(−)) m/e 334 (M−H)⁻; ¹H NMR (300 MHz, DMSO-d₆) δ 7.66 (d, 1H), 7.36 (t, 1H), 7.30-7.26 (m, 2H), 7.22 (m, 1H), 7.06-7.02 (m, 2H), 3.73 (s, 3H), 2.45 (q, 2H), 1.09 (t, 3H).

### EXAMPLE 34

### 2-{[(3-chloro-4-fluorophenyl)sulfonyl]amino}-5-ethylbenzoic Acid

The desired product was prepared by substituting 3-chloro-4-fluorobenzenesulfonyl chloride for benzenesulfonyl chloride in Example 1D. MS (ESI(+)) m/e 358, 360 (M+H)⁺, 375, 377 (M+NH₄)⁺, 380, 382 (M+Na)⁺; (ESI(−)) m/e 356, 358 (M−H)⁻; ¹H NMR (300 MHz, DMSO-d₆) δ 7.81 (dd, 1H), 7.70 (m, 1H), 7.66 (d, 1H), 7.49 (t, 1H), 7.22 (d, 1H), 7.07 (dd, 1H), 2.46 (q, 2H), 1.10 (t, 3H).

### EXAMPLE 35

### 2-{[(2,5-dimethoxyphenyl)sulfonyl]amino}-5-ethylbenzoic Acid

The desired product was prepared by substituting 2,5-dimethoxybenzenesulfonyl chloride for benzenesulfonyl chloride in Example 1D. MS (ESI(+)) m/e 366 (M+H)⁺, 383 (M+NH₄)⁺, 388 (M+Na)⁺; (ESI(−)) m/e 364 (M−H)⁻; ¹H NMR (300 MHz, DMSO-d₆) δ 7.67 (d, 1H), 7.31 (d, 1H), 7.21 (d, 1H), 7.02 (dd, 1H), 6.98 (d, 1H), 6.95 (dd, 1H), 3.70 (s, 3H), 3.65 (s, 3H), 2.44 (q, 2H), 1.08 (t, 3H).

### EXAMPLE 36

### 5-ethyl-2-{[(5-fluoro-2-methylphenyl)sulfonyl]amino}benzoic Acid

The desired product was prepared by substituting 2-methyl-5-fluorobenzenesulfonyl chloride for benzenesulfonyl chloride in Example 1D. MS (ESI(+)) m/e 338 (M+H)⁺, 355 (M+NH₄)⁺, 360 (M+Na)⁺; (ESI(−)) m/e 336 (M−H)⁻; ¹H NMR (300 MHz, DMSO-d₆) δ 7.66 (d, 1H), 7.64 (dd, 1H), 7.29 (dd, 1H), 7.23 (td, 1H), 7.11 (d, 1H), 7.01 (dd, 1H), 2.50 (s, 3H), 2.44 (q, 2H), 1.08 (t, 3H).

### EXAMPLE 37

### 5-ethyl-2-[(8-quinolinylsulfonyl)amino]benzoic Acid

The desired product was prepared by substituting 8-chlorosulfonylquinoline for benzenesulfonyl chloride in Example 1D. MS (ESI(+)) m/e 357 (M+H)⁺, 379 (M+NH₄)⁺; (ESI(−)) m/e 356 (M−H)⁻; ¹H NMR (300 MHz, DMSO-d₆) δ 8.97 (dd, 1H), 8.46-8.42 (m, 2H), 8.22 (d, 1H), 7.71 (t, 1H), 7.64 (dd, 1H), 7.58 (d, 1H), 7.43 (d, 1H), 7.09 (dd, 1H), 2.41 (q, 2H), 1.03 (t, 3H).

### EXAMPLE 38

### 5-ethyl-2-{[2-(methylsulfonyl)phenyl]sulfonyl}amino)benzoic Acid

The desired product was prepared by substituting 2-(methylsulfonyl)benzenesulfonyl chloride for benzenesulfonyl chloride in Example 1D. MS (ESI(+)) m/e 384 (M+H)⁺, 401 (M+NH₄)⁺, 406 (M+Na)⁺; (ESI(−)) m/e 382 (M−H)⁻; ¹H NMR (300 MHz, DMSO-d₆) δ 8.17 (dd, 1H), 8.04 (dd, 1H), 7.78-7.70 (m, 3H), 7.10 (d, 1H), 7.01 (dd, 1H), 2.50 (s, 3H), 2.46 (q, 2H), 1.09 (t, 3H).

### EXAMPLE 39

### 5-ethyl-2-({[2-(trifluoromethoxy)phenyl]sulfonyl}amino)benzoic Acid

The desired product was prepared by substituting 2-(trifluoromethoxy)benzenesulfonyl chloride for benzenesulfonyl chloride in Example 1D. MS (ESI(+)) m/e 390 (M+H)⁺, 407 (M+NH₄)⁺, 412 (M+Na)⁺; (ESI(−)) m/e 388 (M−H)⁻; ¹H NMR (300 MHz, DMSO-d₆) δ 7.96 (dd, 1H), 7.66 (d, 1H), 7.58 (td, 1H), 7.43 (t, 1H), 7.39 (d, 1H), 7.15 (d, 1H), 7.01 (dd, 1H), 2.44 (q, 2H), 1.08 (t, 3H).

### EXAMPLE 40

### 2-({[5-(dimethylamino)-1-naphthyl]sulfonyl}amino)-5-ethylbenzoic Acid

The desired product was prepared by substituting 5-(dimethylamino)-1-naphthalenesulfonyl chloride for benzenesulfonyl chloride in Example 1D. MS (ESI(+)) m/e 399 (M+H)⁺, 421 (M+Na)⁺; (ESI(−)) m/e 398 (M−H)⁻; ¹H NMR (300 MHz, DMSO-d₆) δ 8.42 (d, 1H), 8.34 (d, 1H), 8.19 (d, 1H), 7.60 (d, 1H), 7.55 (t, 1H), 7.50 (t, 1H), 7.17 (d, 1H), 7.15 (d, 1H), 6.97 (dd, 1H), 2.78 (s, 6H), 2.40 (q, 2H), 1.04 (t, 3H).

### EXAMPLE 41

### 2-({[3,5-bis(trifluoromethyl)phenyl]sulfonyl}amino)-5-ethylbenzoic Acid

The desired product was prepared by substituting 3,5-di(trifluoromethyl)benzenesulfonyl chloride for benzenesulfonyl chloride in Example 1D. MS (ESI(+)) m/e 442 (M+H)⁺, 459 (M+NH₄)⁺, 464 (M+Na)⁺; (ESI(−)) m/e 440 (M−H)⁻; ¹H NMR (300 MHz, DMSO-d₆) δ 8.36 (s, 1H), 8.20 (s, 2H), 7.67 (d, 1H), 7.33 (d, 1H), 7.27 (d, 1H), 2.53 (q, 2H), 1.10 (t, 3H).

### EXAMPLE 42

### 2-[(phenylsulfonyl)amino]-1-naphthoic Acid

### EXAMPLE 42A

### 1H-benzo[e]indole-1,2(3H)-dione

A mixture of 2-naphthylamine (8.0 g, 56 mmol) in glacial acetic acid (500 mL) was treated with diethyl ketomalonate (9.2 mL, 62 mmol), heated to 120° C. for 4 hours, and concentrated. The concentrate was suspended in a solution of KOH (36.8 g, 690 mmol) in water (736 mL) and stirred overnight with a stream of air blowing into the solution. The resulting mixture was filtered and the filtrate was adjusted to approximately pH 3 with concentrated HCl. The resulting suspension was cooled to 0° C. and filtered. The filter cake was dried under vacuum to provide the desired product (8.76 g, 79%). MS (DCI) m/e 198 (M+H)⁺, 215 (M+NH₄)⁺.

### EXAMPLE 42B

### 2-amino-1-naphthoic Acid

The desired product was prepared by substituting Example 42A for Example 1B in Example 1C. MS (ESI) m/e 200 (M−H)⁻.

### EXAMPLE 42C

### 2-[(phenylsulfonyl)amino]-1-naphthoic Acid

A mixture of Example 42B (0.033 g, 0.200 mmol) in dichloromethane (1 mL) was treated with 1M chlorotrimethylsilane in dichloromethane (440 μL, 0.044 mmol) and pyridine (56.6 μL, 0.70 mmol), shaken for 4 hours at ambient temperature, treated with a solution of benzenesulfonyl chloride (0.042 g, 0.24 mmol) in dimethylacetamide (1 mL), shaken for 16 hours at ambient temperature, and concentrated. The concentrate was acidified to pH 1.0 with 5% aqueous HCl and extracted with dichloromethane. The extracts were washed sequentially with water and brine, dried (MgSO₄), filtered, and concentrated. The concentrate was purified by C₁₈ reverse-phase HPLC using acetonitrile/water/0.1% TFA to provide the desired product. MS (ESI(+)) m/e 328 (M+H)⁺, 345 (M+NH₄)⁺, 350 (M+Na)⁺; (ESI(−)) m/e 326 (M−H)⁻; ¹H NMR (300 MHz, DMSO-d₆) δ 10.30 (br s, 1H), 8.10 (d, 1H), 7.95 (d, 1H), 7.89 (d, 1H), 7.78 (dd, 2H), 7.63-7.50 (m, 5H), 7.31 (d, 1H).

### EXAMPLE 43

### 2-{[(4-chlorophenyl)sulfonyl]amino}-1-naphthoic Acid

The desired product was prepared by substituting 4-chlorobenzenesulfonyl chloride for benzenesulfonyl chloride in Example 42C. MS (ESI(+)) m/e 379, 381 (M+NH₄)⁺, 384, 386 (M+Na)⁺; (ESI(−)) m/e 360, 362 (M−H)⁻; ¹H NMR (300 MHz, DMSO-d₆) δ 10.36 (br s, 1H), 8.05 (d, 1H), 7.96 (d, 1H), 7.75 (d, 2H), 7.63 (d, 2H), 7.59-7.50 (m, 2H), 7.28 (d, 1H).

### EXAMPLE 44

### 2-{[(4-iodophenyl)sulfonyl]amino}-1-naphthoic Acid

The desired product was prepared by substituting 4-iodobenzenesulfonyl chloride for benzenesulfonyl chloride in Example 42C. MS (ESI(+)) m/e 471 (M+NH₄)⁺, 475.9 (M+Na)⁺; (ESI(−)) m/e 451.9 (M−H)⁻; ¹H NMR (300 MHz, DMSO-d₆) δ 8.12 (br d, 1H), 7.98-7.89 (m, 4H), 7.6-7.52 (m, 2H), 7.5 (d, 2H), 7.3 (d, 2H).

### EXAMPLE 45

### 2-[(1−naphthylsulfonyl)amino]-1-naphthoic Acid

The desired product was prepared by substituting 1-naphthalenesulfonyl chloride for benzenesulfonyl chloride in Example 42C. MS (ESI(+)) m/e 395 (M+NH₄)⁺, 400 (M+Na)⁺; (ESI(−)) m/e 376 (M−H)⁻; ¹H NMR (300 MHz, DMSO-d₆) δ 8.7 (d, 1H), 8.2 (m, 2H), 8.08 (d, 1H), 7.82 (d, 2H), 7.75-7.4 (m, 6H), 7.24 (d, 1H).

### EXAMPLE 46

### 2-{[(3-fluorophenyl)sulfonyl]amino 1-1-naphthoic Acid

The desired product was prepared by substituting 3-fluorobenzenesulfonyl chloride for benzenesulfonyl chloride in Example 42C. MS (ESI(+)) m/e 363 (M+NH₄)⁺, 368 (M+Na)⁺; (ESI(−)) m/e 344 (M−H)⁻; ¹H NMR (300 MHz, DMSO-d₆) δ 8.13 (br d, 1H), 7.98-7.89 (m, 2H), 7.65-7.46 (m, 6H), 7.3 (d, 1H).

### EXAMPLE 47

### 2-{[(4-fluorophenyl)sulfonyl]amino 1-1-naphthoic Acid

The desired product was prepared by substituting 4-fluorobenzenesulfonyl chloride for benzenesulfonyl chloride in Example 42C. MS (ESI(+)) m/e 363 (M+NH₄)⁺, 368 (M+Na)⁺; (ESI(−)) m/e 344 (M−H)⁻; ¹H NMR (300 MHz, DMSO-d₆) δ 8.1 (br d, 1H), 7.98-7.89 (m, 2H), 7.85-7.75 (m, 2H), 7.62-7.49 (m, 2H), 7.43-7.31 (m, 3H).

### EXAMPLE 48

### 2-{[(3,4-difluorophenyl)sulfonyl]amino}-1-naphthoic Acid

The desired product was prepared by substituting 3,4-difluorobenzenesulfonyl chloride for benzenesulfonyl chloride in Example 42C. MS (ESI(+)) m/e 381 (M+NH₄)⁺, 386 (M+Na)⁺; (ESI(−)) m/e 362 (M−H)⁻; ¹H NMR (300 MHz, DMSO-d₆) δ 7.89-7.7 (m, 4H), 7.63-7.5 (m, 3H), 7.47 (m, 1H), 7.35 (m, 1H).

### EXAMPLE 49

### 2-{[(2-chloro-4-fluorophenyl)sulfonyl]amino}-1-naphthoic Acid

The desired product was prepared by substituting 2-chloro-4-fluorobenzenesulfonyl chloride for benzenesulfonyl chloride in Example 42C. MS (ESI(+)) m/e 397, 399 (M+NH₄)⁺, 402, 404 (M+Na)⁺; (ESI(−)) m/e 378, 380 (M−H)⁻; ¹H NMR (300 MHz, DMSO-d₆) δ 8.24 (s, 1H), 8.04 (dd, 1H), 7.95 (d, 1H), 7.89 (d, 1H), 7.68 (dd, 1H), 7.58 (t, 1H), 7.50 (t, 1H), 7.42 (d, 1H), 7.365 (td, 1H).

### EXAMPLE 50

### 2-{[(2-methylphenyl)sulfonyl]amino}-1-naphthoic Acid

The desired product was prepared by substituting 2-methylbenzenesulfonyl chloride for benzenesulfonyl chloride in Example 42C. MS (ESI(+)) m/c 342 (M+H)⁺, 359 (M+NH₄)⁺, 364 (M+Na)⁺; (ESI(−)) m/e 340 (M−H)⁻; ¹H NMR (300 MHz, DMSO-d₆) δ7.93 (t, 1H), 7.87 (d, 1H), 7.83 (d, 1H), 7.65 (d, 1H), 7.56 (m, 1H), 7.51-7.47 (m, 2H), 7.42-7.37 (m, 2H), 7.32 (m, 1H), 2.59 (s, 3H).

### EXAMPLE 51

### 2-{[(3-methylphenyl)sulfonyl]amino}-1-naphthoic Acid

The desired product was prepared by substituting 3-methylbenzenesulfonyl chloride for benzenesulfonyl chloride in Example 42C. MS (ESI(+)) m/e 342 (M+H)⁺, 359 (M+NH₄)⁺, 364 (M+Na)⁺; (ESI(−)) m/e 340 (M−H)⁻; ¹H NMR (300 MHz, DMSO-d₆) δ 8.13 (d, 1H), 7.95 (d, 1H), 7.89 (d, 1H), 7.62 (s, 1H), 7.59-7.55 (m, 2H), 7.51 (td, 1H), 7.44-7.40 (m, 2H), 7.35 (d, 1H), 2.34 (s, 3H).

### EXAMPLE 52

### 2-{[(4-methylphenyl)sulfonyl]amino}-1-naphthoic Acid

The desired product was prepared by substituting 4-methylbenzenesulfonyl chloride for benzenesulfonyl chloride in Example 42C. MS (ESI(+)) m/e 342 (M+H)⁺, 359 (M+NH₄)⁺, 364 (M+Na)⁺; (ESI(−)) m/e 340 (M−H)⁻; ¹H NMR (300 MHz, DMSO-d₆) δ 8.17 (d, 1H), 7.94 (d, 1H), 7.88 (d, 1H), 7.66 (d, 2H), 7.57 (m, 1H), 7.49 (m, 1H), 7.37 (d, 1H), 7.33 (d, 2H), 2.33 (s, 3H).

### EXAMPLE 53

### 2-{[(2-fluorophenyl)sulfonyl]amino}-1-naphthoic Acid

The desired product was prepared by substituting 2-fluorobenzenesulfonyl chloride for benzenesulfonyl chloride in Example 42C. MS (ESI(+)) m/e 346 (M+H)⁺, 363 (M+NH₄)⁺, 368 (M+Na)⁺; (ESI(−)) n/e 344 (M−H)⁻; ¹H NMR (300 MHz, DMSO-d₆) δ 8.20 (m, 1H), 7.96 (d, 1H), 7.89 (d, 1H), 7.77 (td, 1H), 7.67 (m, 1H), 7.57 (t, 1H), 7.50 (t, 1H), 7.45 (d, 1H), 7.38 (t, 1H), 7.31 (t, 1H).

### EXAMPLE 54

### 2-{[(5-fluoro-2-methylphenyl)sulfonyl]amino}-1-naphthoic Acid

The desired product was prepared by substituting 5-fluoro-2-methylbenzenesulfonyl chloride for benzenesulfonyl chloride in Example 42C. MS (ESI(+)) m/e 360 (M+H)⁺, 377 (M+NH₄)⁺, 382 (M+Na)⁺; (ESI(−)) m/e 375 (M−H)⁻; ¹H NMR (300 MHz, DMSO-d₆) δ 8.12 (d, 1H), 7.96 (d, 1H), 7.91 (d, 1H), 7.60-7.50 (m, 3H), 7.44 (dd, 1H), 7.40 (dd, 1H), 7.36 (d, 1H), 2.53 (s, 3H).

### EXAMPLE 55

### 2-{[(2-methoxy-5-methylphenyl)sulfonyl]amino})-1-naphthoic Acid

The desired product was prepared by substituting 2-methoxy-5-methylbenzenesulfonyl chloride for benzenesulfonyl chloride in Example 42C. MS (ESI(+)) m/e 372 (M+H)⁺, 389 (M+NH₄)⁺, 394 (M+Na)⁺; (ESI(−)) m/e 370 (M−H)⁻; ¹H NMR (300 MHz, DMSO-d₆) δ 8.30 (d, 1H), 7.97 (d, 1H), 7.85 (d, 1H), 7.69 (d, 1H), 7.61 (d, 1H), 7.57 (td, 1H), 7.46 (t, 1H), 7.36 (dd, 1H), 7.05 (d, 1H), 3.83 (s, 3H), 2.23 (s, 3H).

### EXAMPLE 56

### 2-{[(2-chloro-6-methylphenyl)sulfonyl]amino}-1-naphthoic Acid

The desired product was prepared by substituting 2-chloro-6-methylbenzenesulfonyl chloride for benzenesulfonyl chloride in Example 42C. MS (ESI(+)) m/e 376, 378 (M+H)⁺, 393, 395 (M+NH₄)⁺, 398, 400 (M+Na)⁺; (ESI(−)) m/e 374, 376 (M−H)⁻; ¹H NMR (300 MHz, DMSO-d₆) δ 8.25 (d, 1H), 7.98 (d, 1H), 7.88 (d, 1H), 7.58 (td, 1H), 7.50 (m, 1H), 7.48-7.42 (m, 3H), 7.35 (dd, 1H), 2.60 (s, 3H).

### EXAMPLE 57

### 2-[(8-quinolinylsulfonyl)amino]-1-naphthoic Acid

The desired product was prepared by substituting 8-(chlorosulfonyl)quinoline for benzenesulfonyl chloride in Example 42C. MS (ESI(+)) m/e 379 (M+H)⁺, 401 (M+Na)⁺; (ESI(−)) m/e 377 (M−H)⁻; ¹H NMR (300 MHz, DMSO-d₆) δ 9.11 (dd, 1H), 8.50 (dd, 1H), 8.43 (dd, 1H), 8.27 (dd, 1H), 8.06 (d, 1H), 7.93 (d, 1H), 7.82-7,78 (m, 2H), 7.74-7.70 (m, 2H), 7.49 (td, 1H), 7.40 (t, 1H).

### EXAMPLE 58

### 2-({[2-(trifluoromethoxy)phenyl]sulfonyl}amino)-1-naphthoic Acid

The desired product was prepared by substituting 2-(trifluoromethoxy)benzenesulfonyl chloride for benzenesulfonyl chloride in Example 42C. MS (ESI(+)) m/e 412 (M+H)⁺, 429 (M+NH₄)⁺, 435 (M+Na)⁺; (ESI(−)) m/e 410 (M−H)⁻; ¹H NMR (300 MHz, DMSO-d₆) δ 8.28 (m, 1H), 7.97 (m, 2H), 7.88 (d, 1H), 7.75 (t, 1H), 7.57 (t, 1H), 7.53-7.46 (m, 4H).

### EXAMPLE 59

### 2-{[(3,5-dichloro-2-hydroxyphenyl)sulfonyl]amino}-1-naphthoic Acid

The desired product was prepared by substituting 3,5-dichloro-2-hydroxybenzenesulfonyl chloride for benzenesulfonyl chloride in Example 42C. MS (ESI(+)) m/e 429, 431 (M+NH₄)⁺, 434, 436 (M+Na)⁺; (ESI(−)) m/e 427, 429 (M−H)⁻; ¹H NMR (300 MHz, DMSO-d₆) δ 8.23 (d, 1H), 7.99 (d, 1H), 7.89 (d, 1H), 7.83 (d, 1H), 7.66 (d, 1H), 7.59 (td, 1H), 7.51 (d, 1H), 7.48 (d, 1H).

### EXAMPLE 60

### 2-{[4-chloro-3-(trifluoromethyl)phenyl] sulfonyl}amino)-1-naphthoic Acid

The desired product was prepared by substituting 4-chloro-3-(trifluoromethyl)benzenesulfonyl chloride for benzenesulfonyl chloride in Example 42C. MS (ESI(+)) m/e 452, 454 (M+H)⁺, 469, 471 (M+NH₄)⁺, 474, 476 (M+Na)⁺; (ESI(−)) m/e 450, 452 (M−H)⁻; ¹H NMR (300 MHz, DMSO-d₆) δ 8.07 (s, 1H), 8.01-7.89 (m, 5H), 7.59 (t, 1H), 7.53 (t, 1H), 7.33 (t, 1H).

### EXAMPLE 61

### 2-[({2-[(3-aminopropyl)amino]phenyl}sulfonyl)amino]-1-naphthoic Acid

### EXAMPLE 61A

### 2-{[(2-bromophenyl)sulfonyl]amino}-1-naphthoic Acid

The desired product was prepared by substituting 2-bromobenzenesulfonyl chloride for benzenesulfonyl chloride in Example 42C. MS m/e 405 (M−H)⁻.

### EXAMPLE 61B

### 2-[({2-[(3-aminopropyl)amino]phenyl}sulfonyl)amino]-1-naphthoic Acid

A mixture of Example 61A (90 mg, 0.22 mmol) in N,N-dimethylformamide (1 mL) was treated with ethylene diamine (1 mL), heated to reflux for 2 days, and dried under vacuum. The concentrate was purified by C₁₈ reverse-phase HPLC with acetonitrile/water/0.1% TFA to provide the desired product. MS (ESI(−)) m/e 398 (M−H)⁻; ¹H NMR (300 MHz, DMSO-d₆) δ 10.53 (s, 1H), 8.05 (m, 1H), 7.96 (dd, 1H), 7.70-7.82 (m.3H), 7.65 (d, 1H), 7.21-7.51 (m, 4H), 6.73 (d, 1H), 6.57 (t, 1H), 3.01-3.26 (m, 4H).

### EXAMPLE 62

### 2-{[(2,4-dimethoxyphenyl)sulfonyl]amino}-1-naphthoic Acid

The desired product was prepared by substituting 2,4-dimethoxybenzenesulfonyl chloride for benzenesulfonyl chloride in Example 42C. MS (ESI(+)) m/e 388 (M+H)⁺, 405 (M+NH₄)⁺, 410 (M+Na)⁺; (ESI(−)) m/e 386 (M−H)⁻; ¹H NMR (300 MHz, DMSO-d₆) δ 9.20 (d, 1H), 7.73 (d, 1H), 7.65-7.58 (m, 3H), 7.35 (m, 2H), 7.22 (td, 1H), 6.52 (dd, 1H), 6.50 (s, 1H), 3.76 (s, 6H).

### EXAMPLE 63

### 2-{[(4-methoxyphenyl)sulfonyl]amino}-1-naphthoic Acid

The desired product was prepared by substituting 4-methoxybenzenesulfonyl chloride for benzenesulfonyl chloride in Example 42C. MS (ESI(+)) m/e 358 (M+H)⁺, 380 (M+Na)⁺; (ESI(−)) m/e 356 (M−H)⁻; ¹H NMR (300 MHz, DMSO-d₆) δ 10.25 (br s, 1H), 8.14 (d, 1H), 7.95 (d, 1H), 7.90 (d, 1H), 7.70 (dt, 2H), 7.54 (m, 2H), 7.39 (d, 1H), 7.05 (dt, 1H), 3.79 (s, 3H).

### EXAMPLE 64

### 2-[(butylsulfonyl)amino]-5-ethylbenzoic Acid

The desired product was prepared by substituting 1-butanesulfonyl chloride for benzenesulfonyl chloride in Example 1D. MS (ESI(+)) m/e 286 (M+H)⁺, 303 (M+NH₄)⁺, 308 (M+Na)⁺; (ESI(−)) m/e 284 (M−H)⁻; ¹H NMR (300 MHz, DMSO-d₆) δ 7.77 (d, 1H), 7.32 (d, 1H), 7.11 (dd, 1H), 2.92 (t, 2H), 2.52 (q, 2H), 1.56 (m, 2H), 1.28 (m, 2H), 1.15 (t, 3H), 0.99 (t, 3H).

### EXAMPLE 65

### 5-ethyl-2-[(2-thienylsulfonyl)amino]benzoic Acid

The desired product was prepared by substituting 2-thiophenesulfonyl chloride for benzenesulfonyl chloride in Example 1D. MS (ESI(+)) m/e 312 (M+H)⁺, 329 (M+NH₄)⁺, 334 (M+Na)⁺; (ESI(−)) m/e 310 (M−H)⁻; ¹H NMR (300 MHz, DMSO-d₆) δ 7.67-7.65 (m, 2H), 7.38 (dd, 1H), 7.31 (d, 1H), 7.07 (m, 1H), 6.99 (dd, 1H), 2.47 (q, 2H), 1.10 (t, 3H).

### EXAMPLE 66

### 2-{[(5-chloro-1,3-dimethyl-1H-pyrazol-4-yl)sulfonyl]amino}-5-ethylbenzoic Acid

The desired product was prepared by substituting 5-chloro-1,3-dimethyl-1H-pyrazole-4-sulfonyl chloride for benzenesulfonyl chloride in Example 1D. MS (ESI(+)) m/e 358, 360 (M+H)⁺, 375, 377 (M+NH₄)⁺, 380, 382 (M+Na)⁺; (ESI(−)) m/e 356, 358 (M−H)⁻; ¹H NMR (300 MHz, DMSO-d₆) δ 7.67 (d, 1H), 7.17 (d, 1H), 7.02 (dd, 1H), 3.67 (s, 3H), 2.46 (q, 2H), 2.24 (s, 3H), 1.10 (t, 3H).

### EXAMPLE 67

### 5-ethyl-2-({[2-(methoxycarbonyl)-3-thienyl]sulfonyl} amino)benzoic Acid

The desired product was prepared by substituting methyl 3-(chlorosulfonyl)-2-thiophenecarboxylate for benzenesulfonyl chloride in Example 1D. MS (ESI(+)) m/e 370 (M+H)⁺, 387 (M+NH₄)⁺, 392 (M+Na)⁺; (ESI(−)) m/e 368 (M−H)⁻; ¹H NMR (300 MHz, DMSO-d₆) δ 7.81 (d, 1H), 7.69 (d, 1H), 7.39 (d, 1H), 7.19 (d, 1H), 7.01 (dd, 1H), 3.81 (s, 3H), 2.46 (q, 2H), 1.10 (t, 3H).

### EXAMPLE 68

### 2-[(2,1,3-benzothiadiazol-4-ylsulfonyl)amino]-1-naphthoic Acid

The desired product was prepared by substituting 2,1,3-benzothiadiazole-4-sulfonyl chloride for benzenesulfonyl chloride in Example 42C. MS (ESI(+)) m/e 403 (M+NH₄)⁺, 408 (M+Na)⁺; (ESI(−)) m/e 384 (M−H)⁻; ¹H NMR (300 MHz, DMSO-d₆) δ 8.36 (d, 1H), 8.22 (d, 1H), 7.99 (d, 1H), 7.96 (d, 1H), 7.87 (dd, 1H), 7.78 (dd, 1H), 7.60 (d, 1H), 7.55-7.44 (m, 2H).

### EXAMPLE 69

### 2-[(butylsulfonyl)amino]-1-naphthoic Acid

The desired product was prepared by substituting 1-butanesulfonyl chloride for benzenesulfonyl chloride in Example 42C. MS (ESI(+)) m/e 325 (M+NH₄)⁺, 330 (M+Na)⁺; (ESI(−)) m/e 306 (M−H)⁻; ¹H NMR (300 MHz, DMSO-d₆) δ 8.17 (d, 1H), 8.04 (d, 1H), 7.95 (d, 1H), 7.66 (d, 1H), 7.61 (td, 1H), 7.53 (t, 1H), 3.19 (m, 2H), 1.68 (m, 2H), 1.36 (m, 2H), 0.84 (t, 3H).

### EXAMPLE 70

### 2-[(2-thienylsulfonyl)amino]-1-naphthoic Acid

The desired product was prepared by substituting 2-thiophenesulfonyl chloride for benzenesulfonyl chloride in Example 42C. MS (ESI(+)) m/e 351 (M+NH₄)⁺, 356 (M+Na)⁺; (ESI(−)) m/e 332 (M−H)⁻; ¹H NMR (300 MHz, DMSO-d₆) δ 8.36 (d, 1H), 7.96 (d, 1H), 7.89-7.84 (m, 2H), 7.56 (t, 1H), 7.51 (d, 1H), 7.50-7.42 (m, 2H), 7.10 (t, 1H).

### EXAMPLE 71

### 2-[(benzylsulfonyl)amino]-1-naphthoic Acid

The desired product was prepared by substituting phenylmethanesulfonyl chloride for benzenesulfonyl chloride in Example 42C. MS (ESI(+)) m/e 359 (M+NH₄)⁺, 364 (M+Na)⁺; (ESI(−)) m/e 340 (M−H)⁻; ¹H NMR (300 MHz, DMSO-d₆) δ 8.28 (d, 1H), 7.98 (d, 1H), 7.93 (d, 1H), 7.60 (t, 1H), 7.57-7.49 (m, 2H), 7.34 (m, 5H), 4.59 (s, 2H).

### EXAMPLE 72

### 2-{[(3,5-dimethyl-4-isoxazolyl)sulfonyl]amino}-1-naphthoic Acid

The desired product was prepared by substituting 3,5-dimethyl-4-isoxazolesulfonyl chloride for benzenesulfonyl chloride in Example 42C. MS (ESI(+)) m/e 364 (M+NH₄)⁺, 369 (M+Na)⁺; (ESI(−)) m/e 346 (M−H)⁻; ¹H NMR (300 MHz, DMSO-d₆) δ 8.06 (d, 1H), 8.04 (d, 1H), 7.96 (d, 1H), 7.61 (m, 1H), 7.57 (td, 1H), 7.47 (d, 1H), 2.31 (s, 3H), 2.14 (s, 3H).

### EXAMPLE 73

### 2-({[(E)-2-phenylvinyl]sulfonyl}amino)-1-naphthoic Acid

The desired product was prepared by substituting (E)-2-phenylethylenesulfonyl chloride for benzenesulfonyl chloride in Example 42C. MS (ESI(+)) m/e 354 (M+H)⁺, 371 (M+NH₄)⁺, 376 (M+Na)⁺; (ESI(−)) m/e 352 (M−H)⁻; ¹H NMR (300 MHz, DMSO-d₆) δ 8.18 (d, 1H), 8.02 (d, 1H), 7.93 (d, 1H), 7.66 (m, 2H), 7.64 (d, 1H), 7.59 (td, 1H), 7.51 (t, 1H), 7.45 (d, 1H), 7.42-7.39 (m, 3H), 7.32 (d, 1H).

### EXAMPLE 74

### 2-{[(5-chloro-2-thienyl)sulfonyl]amino}-1-naphthoic Acid

The desired product was prepared by substituting 5-chloro-2-thiophenesulfonyl chloride for benzenesulfonyl chloride in Example 42C. MS (ESI(+)) m/e 385, 387 (M+NH₄)⁺, 390, 392 (M+Na)⁺; (ESI(−)) m/e 366, 368 (M−H)⁻; ¹H NMR (300 MHz, DMSO-d₆) δ 8.11 (d, 1H), 8.02 (d, 1H), 7.95 (d, 1H), 7.61 (t, 1H), 7.55 (t, 1H), 7.39 (d, 1H), 7.37 (d, 1H), 7.18 (d, 1H).

### EXAMPLE 75

### 2-{[(5-chloro-1,3-dimethyl-1H-pyrazol-4-yl)sulfonyl]amino}-1-naphthoic Acid

The desired product was prepared by substituting 5-chloro-1,3-dimethyl-1H-pyrazole-4-sulfonyl chloride for benzenesulfonyl chloride in Example 42C. MS (ESI(+)) m/e 380, 382 (M+H)⁺, 397, 399 (M+N₄)⁺, 402, 404 (M+Na)⁺; (ESI(−)) m/e 378, 380 (M−H)⁻; ¹H NMR (300 MHz, DMSO-d₆) δ 8.16 (d, 1H), 8.04 (d, 1H), 7.94 (d, 1H), 7.59 (td, 1H), 7.55 (d, 1H), 7.53 (t, 1H), 3.69 (s, 3H), 2.11 (s, 3H).

### EXAMPLE 76

### 2-({[2-(methoxycarbonyl)-3-thienyl]sulfonyl}amino)-1-naphthoic Acid

The desired product was prepared by substituting methyl 3-(chlorosulfonyl)-2-thiophenecarboxylate for benzenesulfonyl chloride in Example 42C. MS (ESI(+)) m/e 392 (M+H)⁺, 409 (M+NH₄)⁺, 414 (M+Na)⁺; (ESI(−)) m/e 390 (M−H)⁻; ¹H NMR (300 MHz, DMSO-d₆) δ 8.14 (d, 1H), 7.99 (d, 1H), 7.97 (d, 1H), 7.90 (d, 1H), 7.63 (d, 1H), 7.58 (td, 1H), 7.51-7.48 (m, 2H), 3.90 (s, 3H).

### EXAMPLE 77

### 2-({[5-(3-isoxazolyl)-2-thienyl]sulfonyl}amino)-1-naphthoic Acid

The desired product was prepared by substituting 5-(3-isoxazolyl)-2-thiophenesulfonyl chloride for benzenesulfonyl chloride in Example 42C. MS (ESI(+)) m/e 418 (M+NH₄)⁺, 423 (M+Na)⁺; (ESI(−)) m/e 399 (M−H)⁻; ¹H NMR (300 MHz, DMSO-d₆) δ 8.70 (d, 1H), 8.11 (d, 1H), 8.01 (d, 1H), 7.94 (d, 1H), 7.67 (d, 1H), 7.60 (td, 1H), 7.56-7.53 (m, 2H), 7.41 (d, 1H), 7.06 (d, 1H).

### EXAMPLE 78

### 2-{[(2,5-dichloro-3-thienyl)sulfonyl]amino}-1-naphthoic Acid

The desired product was prepared by substituting 2,5-dichloro-3-thiophenesulfonyl chloride for benzenesulfonyl chloride in Example 42C. MS (ESI(+)) m/e 419, 421 (M+NH₄)⁺, 424, 426 (M+Na)⁺; (ESI(−)) m/e 400, 402 (M−H)⁻; ¹H NMR (300 MHz, DMSO-d₆) δ 8.11 (d, 1H), 8.02 (d, 1H), 7.95 (d, 1H), 7.61 (td, 1H), 7.55 (t, 1H), 7.42 (d, 1H), 7.24 (s, 1H).

### EXAMPLE 79

### 2-{[(4,5-dichloro-2-thienyl)sulfonyl]amino}-1-naphthoic Acid

The desired product was prepared by substituting 4,5-dichloro-2-thiophenesulfonyl chloride for benzenesulfonyl chloride in Example 42C. MS (ESI(+)) m/e 424, 426 (M+Na)⁺; (ESI(−)) m/e 400, 402 (M−H)⁻; ¹H NMR (300 MHz, DMSO-d₆) δ 8.08 (d, 1H), 8.03 (d, 1H), 7.97 (d, 1H), 7.61 (td, 1H), 7.57 (t, 1H), 7.55 (s, 1H), 7.41 (d, 1H).

### EXAMPLE 80

### 2-{[(5-bromo-6-chloro-3-pyridinyl)sulfonyl]amino}-1-naphthoic Acid

The desired product was prepared by substituting 5-bromo-6-chloro-3-pyridinesulfonyl chloride for benzenesulfonyl chloride in Example 42C. MS (ESI(+)) m/e 463, 465 (M+Na)⁺; (ESI(−)) m/e 439, 441 (M−H)⁻; ¹H NMR (300 MHz, DMSO-d₆) δ 8.61 (d, 1H), 8.38 (d, 1H), 8.01-7.96 (m, 3H), 7.60 (td, 1H), 7.56 (t, 1H), 7.40 (d, 1H).

### EXAMPLE 81

### 2-{[(3-chloropropyl)sulfonyl]amino}-1-naphthoic Acid

The desired product was prepared by substituting 3-chloro-1-propanesulfonyl chloride for benzenesulfonyl chloride in Example 42C. MS (ESI(+)) m/e 345, 347 (M+NH₄)⁺, 350, 352 (M+Na)⁺; (ESI(−)) m/e 326, 328 (M−H)⁻; ¹H NMR (300 MHz, DMSO-d₆) δ 8.18 (br s, 1H), 8.03 (d, 1H), 7.96 (d, 1H), 7.63 (d, 1H), 7.61 (td, 1H), 7.53 (t, 1H), 3.73 (t, 2H), 3.33 (m, 2H), 2.17 (m, 2H).

### EXAMPLE 82

### 2-[(methylsulfonyl)amino]-1-naphthoic Acid

The desired product was prepared by substituting methanesulfonyl chloride for benzenesulfonyl chloride in Example 42C. MS (ESI(−)) m/e 264 (M−H)⁻; ¹H NMR (300 MHz, DMSO-d₆) δ 9.34 (d, 1H), 7.67-7.83 (m, 3H), 7.41 (dt, 1H), 7.29 (dt, 1H), 7.07 (m, 2H), 2.86 (s, 3H).

### EXAMPLE 83

### 2-[(ethylsulfonyl)amino]-1-naphthoic Acid

The desired product was prepared by substituting ethanesulfonyl chloride for benzenesulfonyl chloride in Example 42C. MS (ESI(−)) m/e 278 (M−H)⁻; ¹H NMR (300 MHz, DMSO-d₆) δ 9.31 (d, 1H), 7.72-7.82 (m, 3H), 7.41 (dt, 1H), 7.29 (dt, 1H), 2.98 (q, 4H), 1.15 (t, 3H).

### EXAMPLE 84

### 2-[(propylsulfonyl)amino]-1-naphthoic Acid

The desired product was prepared by substituting 1-propanesulfonyl chloride for benzenesulfonyl chloride in Example 42C. MS (ESI(−)) m/e 292 (M−H)⁻; ¹H NMR (300 MHz, DMSO-d₆) δ 9.32 (d, 1H), 7.72-7.81 (m, 3H), 7.41(dt, 1H), 7.29 (dt, 1H), 2.94-2.98. (m, 2H), 1.59-1.71 (m, 2H), 0.87 (t, 3H).

### EXAMPLE 85

### 7-fluoro-2-[(phenylsulfonyl)amino]-1-naphthoic Acid

### EXAMPLE 85A

### 7-fluoro-2-naphthylamine

A suspension of 7-nitro-2-naphthylamine (2.06 g, 11.0 mmol, prepared as described in J. Chem. Soc. 1949, 1187) in dichloromethane (90 mL) and THF (10 mL) at −20° C. was treated with boron trifluoride diethyletherate (2.1 mL, 16.6 mmol), treated dropwise with tert-butyl nitrite (1.6 mL, 13.5 mmol), warmed to ambient temperature over 2 hours, diluted with diethyl ether (100 mL), and filtered. The filter cake was washed with diethyl ether and dried under vacuum to provide the diazonium tetrafluoroborate salt (3.10 g). The salt was suspended in 1,2-dimethylbenzene, heated to 120° C. until gas evolution ceased, and concentrated. The concentrate was dissolved in dichloromethane (95 mL) and methanol (5 mL), treated with stannous chloride (50 g, 270 mmol, added in three portions), stirred for 4 days, diluted with dichloromethane, treated with 1M NaOH (500 mL), and shaken for 30 seconds. The emulsion was filtered through diatomaceous earth (Celite®) and the filtrate was extracted twice with dichloromethane. The combined extracts were dried (MgSO₄), filtered, and concentrated to provide the desired product (1.68 g). MS (DCI) m/e 162 (M+H)⁺; ¹H NMR (300 MHz, DMSO-d₆) δ 7.67 (dd, 1H), 7.59 (d, 1H), 7.22 (dd, 1H), 6.94 (dd, 1H), 6.88 (dd, 1H), 6.76 (d, 1H).

### EXAMPLE 85B

### 2-amino-7-fluoro-1-naphthoic Acid

The desired product was prepared by substituting Example 85A for 2-naphthylamine in Examples 42A and 42B. MS (ESI(−)) m/e 204 (M−H)⁻; ¹H NMR (300 MHz, DMSO-d₆) δ 8.32 (dd, 1H), 7.76-7.69 (m, 2H), 7.08-6.98 (m, 2H).

### EXAMPLE 85C

### 7-fluoro-2-[(phenylsulfonyl)amino]-1-naphthoic Acid

The desired product was prepared by substituting Example 85B for Example 42B in Example 42C. MS (ESI(+)) m/e 363 (M+NH₄)⁺, 368 (M+Na)⁺; (ESI(−)) m/e 344 (M−H)⁻; ¹H NMR (300 MHz, DMSO-d₆) δ 7.79-7.71 (m, 4H), 7.65 (d, 1H), 7.46 (dd, 2H), 7.13 (td, 1H).

### EXAMPLE 86

### 7-fluoro-2-{1[(4-fluorophenyl)sulfonyl]amino 1-1-naphthoic Acid

The desired product was prepared by substituting Example 85B and 4-fluorobenzenesulfonyl chloride for Example 42B and benzenesulfonyl chloride respectively, in Example 42C. MS (ESI(+)) m/e 381 (M+NH₄)⁺, 386 (M+Na)⁺; (ESI(−)) m/e 362 (M−H)⁻; ¹H NMR (300 MHz, DMSO-d₆) δ 7.85-7.72 (m, 5H), 7.64 (d, 1H), 7.28 (t, 2H), 7.13 (td, 1H).

### EXAMPLE 87

### 7-fluoro-2-{[(3-fluorophenyl)sulfonyl]amino}-1-naphthoic Acid

The desired product was prepared by substituting Example 85B and 3-fluorobenzenesulfonyl chloride for Example 42B and benzenesulfonyl chloride, respectively, in Example 42C. MS (ESI(+)) m/e 381 (M+NH₄)⁺, 386 (M+Na)⁺; (ESI(−)) m/e 362 (M−H)⁻; ¹H NMR (300 MHz, DMSO-d₆) δ 7.81 (d, 1H), 7.79 (s, 1H), 7.77 (d, 1H), 7.63 (d, 1H), 7.59 (m, 1H), 7.54-7.49 (m, 2H), 7.34 (td, 1H), 7.17 (td, 1H).

### EXAMPLE 88

### 2-{[(3,4-difluorophenyl)sulfonyl]amino}-7-fluoro-1-naphthoic Acid

The desired product was prepared by substituting Example 85B and 3,4-difluorobenzenesulfonyl chloride for Example 42B and benzenesulfonyl chloride, respectively, in Example 42C. MS (ESI(+)) m/e 399 (M+NH₄)⁺, 404 (M+Na)⁺; (ESI(−)) m/e 380 (M−H)⁻; ¹H NMR (300 MHz, DMSO-d₆) δ 7.81 (d, 1H), 7.79 (s, 1H), 7.77 (d, 1H), 7.63 (d, 1H), 7.59 (m, 1H), 7.54-7.49 (m, 2H), 7.32 (td, 1H), 7.16 (td, 1H).

### EXAMPLE 89

### 2-{[(2.4-difluorophenyl)sulfonyl]amino}-7-fluoro-1-naphthoic Acid

The desired product was prepared by substituting Example 85B and 2,4-difluorobenzenesulfonyl chloride for Example 42B and benzenesulfonyl chloride, respectively, in Example 42C. MS (ESI(+)) m/e 399 (M+NH₄)⁺, 404 (M+Na)⁺; (ESI(−)) m/e 380 (M−H)⁻; ¹H NMR (300 MHz, DMSO-d₆) δ 7.82-7.72 (m, 3H), 7.64 (d, 1H), 7.62-7.57 (m, 1H), 7.54 (dd, 1H), 7.33 (td, 1H), 7.16 (td, 1H).

### EXAMPLE 90

### 2-[(phenylsulfonyl)amino]-5,6,7,8-tetrahydro-1-naphthalenecarboxylic Acid

A mixture of Example 42C (0.087 g, 0.27 mmol), and platinum oxide (0.056 g, 0.25 mmol) in acetic acid (7.5 mL) was shaken in a reactor pressurized with 60 psi of H₂ at 25° C. for 80 hours and filtered. The filtrate was concentrated and the concentrate was purified by C₁₈ reverse-phase HPLC with acetonitrile/water/0.1% trifluoroacetic acid to provide the desired product. MS (ESI(+)) m/e 332 (M+H)⁺, 349 (M+NH₄)⁺, 354 (M+Na)⁺; (ESI(−)) m/e 330 (M−H)⁻; ¹H NMR (300 MHz, DMSO-d₆) δ 7.74 (m, 2H), 7.63 (m, 1H), 7.56 (m, 2H), 6.98 (d, 1H), 6.63 (d, 1H), 2.65 (m, 4H), 1.66 (m, 4H).

### EXAMPLE 91

### 6-bromo-2-{[(4-fluorophenyl)sulfonyl]amino}-1-naphthoic Acid

### EXAMPLE 91A

### 7-bromo-1H-benzo[e]indole-1,2(3H)-dione

A mixture Example 42A (0.50 g, 2.5 mmol) and bromine (154 μL, 3.0 mmol) in of chloroform (20 mL) and DMF (2 mL) was stirred at ambient temperature for 16 hours and filtered. The filter cake was washed with chloroform and dried under vacuum to provide the desired product (0.50 g, 72%). MS (DCI/NH₃) m/e 294 (M+NH₄)⁺; ¹H NMR (300 MHz, DMSO-d₆) δ 11.12 (s, 1H), 8.32 (d, 1H), 8.25 (s, 1H), 8.22 (d, 1H), 7.80 (dd, 1H), 7.24 (d, 1%).

### EXAMPLE 91B

### 2-amino-6-bromo-1-naphthoic Acid

The desired product was prepared by substituting Example 91A for Example 1B in Example 1C. MS (ESI(−)) m/e 265 (M−H)⁻; ¹H NMR (300 MHz, DMSO-d₆) δ 8.48 (d, 1H), 7.90 (d, 1H), 7.71 (d, 1H), 7.50 (dd, 1H), 7.07 (d, 1H).

### EXAMPLE 91C

### 6-bromo-2-{[(4-fluorophenyl)sulfonyl]amino}-1-naphthoic Acid

The desired compound was prepared by substituting Example 91B and 4-fluorobenzenesulfonyl chloride for Example 42B and benzenesulfonyl chloride, respectively, in Example 42C. MS (ESI(−)) m/e 424 (M−H)⁻; ¹H NMR (300 MHz, DMSO-d₆) δ 9.15 (br s, 1H), 7.98 (d, 1H), 7.83-7.75 (m, 3H), 7.63 (d, 1H), 7.53 (dd, 1H), 7.30 (t, 3H).

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of formula (I) 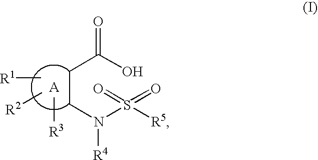
or a therapeutically acceptable salt thereof, wherein
A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur;
R¹, R², and R³ are independently selected from the group consisting of hydrogen, alkoxy, alkoxyalkyl, alkyl, alkylsulfanyl, alkylsulfanylalkyl, amino, aminoalkyl, cycloalkyl, (cycloalkyl)alkyl, halo, haloalkoxy, haloalkyl, and hydroxyalkyl; provided that when A is phenyl, at least one of R¹, R², and R³ is other than hydrogen or C₁ alkyl;
R⁴ is selected from the group consisting of hydrogen, alkyl, alkylsulfanylalkyl, aryl, and arylalkyl; and
R⁵ is selected from the group consisting of alkyl, amino, aminoalkyl, aryl, arylalkenyl, arylalkyl, haloalkyl, heteroaryl, heteroarylalkenyl, heteroarylalkyl, and heterocycle.

2. The compound of claim 1 of formula (II) 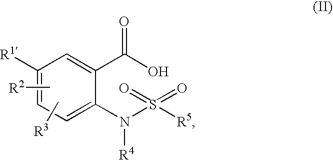
or a therapeutically acceptable salt thereof, wherein
R^{1′} is selected from the group consisting of alkoxy, alkoxyalkyl, C₂-C₁₀ alkyl, alkylsulfanyl, alkylsulfanylalkyl, amino, aminoalkyl, cycloalkyl, (cycloalkyl)alkyl, halo, haloalkoxy, and haloalkyl; and
R², R³, R⁴, and R⁵ are as defined in claim 1.

3. The compound of claim 2 selected from the group consisting of
5-ethyl-2-[(phenylsulfonyl)amino]benzoic acid;
5-isopropyl-2-[(phenylsulfonyl)amino]benzoic acid;
5-isobutyl-2-[(phenylsulfonyl)amino]benzoic acid;
2-[(phenylsulfonyl)amino]-5-propylbenzoic acid;
5-cyclopentyl-2-[(phenylsulfonyl)amino]benzoic acid;
5-cyclohexyl-2-[(phenylsulfonyl)amino]benzoic acid;
5-butyl-2-[(phenylsulfonyl)amino]benzoic acid;
5-(3-methylbutyl)-2-[(phenylsulfonyl)amino]benzoic acid;
5-(2-methylbutyl)-2-[(phenylsulfonyl)amino]benzoic acid;
5-pentyl-2-[(phenylsulfonyl)amino]benzoic acid;
5-(2-ethylbutyl)-2-[(phenylsulfonyl)amino]benzoic acid;
5-hexyl-2-[(phenylsulfonyl)amino]benzoic acid;
2-{[(2-chloro-4-fluorophenyl)sulfonyl]amino}-5-ethylbenzoic acid;
5-ethyl-2-{[(3-methylphenyl)sulfonyl]amino}benzoic acid;
5-ethyl-2-{[(2-fluorophenyl)sulfonyl]amino}benzoic acid;
5-ethyl-2-{[(3-fluorophenyl)sulfonyl]amino}benzoic acid;
5-ethyl-2-{[(4-fluorophenyl)sulfonyl]amino}benzoic acid;
2-{[(2-chlorophenyl)sulfonyl]amino}-5-ethylbenzoic acid;
2-{[(3-chlorophenyl)sulfonyl]amino}-5-ethylbenzoic acid;
2-{[(3,4-difluorophenyl)sulfonyl]amino}-5-ethylbenzoic acid;
5-ethyl-2-[(1−naphthylsulfonyl)amino]benzoic acid;
5-ethyl-2-({[3-(trifluoromethyl)phenyl]sulfonyl}amino)benzoic acid;
2-{[(2,3-dichlorophenyl)sulfonyl]amino}-5-ethylbenzoic acid;
2-{[(2,5-dichlorophenyl)sulfonyl]amino}-5-ethylbenzoic acid;
2-{[(3,5-dichlorophenyl)sulfonyl]amino}-5-ethylbenzoic acid;
2-{[(2-bromophenyl)sulfonyl]amino}-5-ethylbenzoic acid;
2-{[(3-bromophenyl)sulfonyl]amino}-5-ethylbenzoic acid;
5-ethyl-2-{[(4-methylphenyl)sulfonyl]amino}benzoic acid;
2-{[(3-cyanophenyl)sulfonyl]amino}-5-ethylbenzoic acid;
2-{[(4-cyanophenyl)sulfonyl]amino}-5-ethylbenzoic acid;
2-{[(2,5-dimethylphenyl)sulfonyl]amino}-5-ethylbenzoic acid;
5-ethyl-2-{[(3-methoxyphenyl)sulfonyl]amino}benzoic acid;
2-{[(3-chloro-4-fluorophenyl)sulfonyl]amino}-5-ethylbenzoic acid;
2-{[(2,5-dimethoxyphenyl)sulfonyl]amino}-5-ethylbenzoic acid;
5-ethyl-2-{[(5-fluoro-2-methylphenyl)sulfonyl]amino}benzoic acid;
5-ethyl-2-[(8-quinolinylsulfonyl)amino]benzoic acid;
5-ethyl-2-({[2-(methylsulfonyl)phenyl]sulfonyl}amino)benzoic acid;
5-ethyl-2-({[2-(trifluoromethoxy)phenyl]sulfonyl}amino)benzoic acid;
2-({[5-(dimethylamino)-1-naphthyl]sulfonyl}amino)-5-ethylbenzoic acid;
2-({[3,5-bis(trifluoromethyl)phenyl]sulfonyl}amino)-5-ethylbenzoic acid;
2-[(butylsulfonyl)amino]-5-ethylbenzoic acid;
5-ethyl-2-[(2-thienylsulfonyl)amino]benzoic acid;
2-{[(5-chloro-1,3-dimethyl-1H-pyrazol-4-yl)sulfonyl]amino}-5-ethylbenzoic acid; and
5-ethyl-2-({[2-(methoxycarbonyl)-3-thienyl]sulfonyl}amino)benzoic acid.

4. The compound of claim 1 of formula (III) 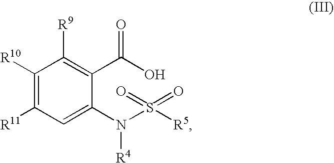
or a therapeutically acceptable salt thereof, wherein
R⁴ and R⁵ are as described in claim 1; and
R⁹ is hydrogen and R₁₀ and R¹¹, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl; or
R¹¹ is hydrogen and R⁹ and R¹⁰, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl.

5. The compound of claim 4 selected from the group consisting of
6-[(phenylsulfonyl)amino]-5-indanecarboxylic acid; and
2-[(phenylsulfonyl)amino]-5,6,7,8-tetrahydro-1-naphthalenecarboxylic acid.

6. A compound of formula (IV) 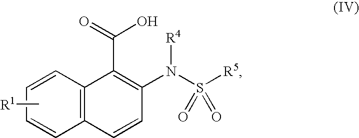
or a therapeutically acceptable salt thereof, wherein
R¹, R⁴, and R⁵ are as defined in claim 1.

7. The compound of claim 6 wherein R⁵ is aryl.

8. The compound of claim 7 wherein R⁵ is aryl wherein the aryl is unsubstituted.

9. The compound of claim 8 selected from the group consisting of
2-[(phenylsulfonyl)amino]-1-naphthoic acid;
2-[(1-naphthylsulfonyl)amino]-1-naphthoic acid; and
7-fluoro-2-[(phenylsulfonyl)amino]-1-naphthoic acid.

10. The compound of claim 7 wherein R⁵ is aryl wherein the aryl is monosubstituted.

11. The compound of claim 10 selected from the group consisting of
2-{[(4-chlorophenyl)sulfonyl]amino}-1-naphthoic acid;
2-{[(4-iodophenyl)sulfonyl]amino}-1-naphthoic acid;
2-{[(3-fluorophenyl)sulfonyl]amino}-1-naphthoic acid;
2-{[(4-fluorophenyl)sulfonyl]amino}-1-naphthoic acid;
2-[(2-methylphenyl)sulfonyl]amino}-1-naphthoic acid;
2-[(3-methylphenyl)sulfonyl]amino}-1-naphthoic acid;
2-[(4-methylphenyl)sulfonyl]amino}-1-naphthoic acid;
2-[(2-fluorophenyl)sulfonyl]amino}-1-naphthoic acid;
2-({[2-(trifluoromethoxy)phenyl]sulfonyl}amino)-1-naphthoic acid;
2-[({2-[(3-aminopropyl)amino]phenyl}sulfonyl)amino]-1-naphthoic acid;
2-{[(4-methoxyphenyl)sulfonyl]amino}-1-naphthoic acid;
7-fluoro-2-{[(4-fluorophenyl)sulfonyl]amino}-1-naphthoic acid;
7-fluoro-2-{[(3-fluorophenyl)sulfonyl]amino}-1-naphthoic acid; and
6-bromo-2-{[(4-fluorophenyl)sulfonyl]amino}-1-naphthoic acid.

12. The compound of claim 7 wherein R⁵ is aryl wherein the aryl is disubstituted or trisubstituted.

13. The compound of claim 12 selected from the group consisting of
2-{[(3,4-difluorophenyl)sulfonyl]amino}-1-naphthoic acid;
2-{[(2-chloro-4-fluorophenyl)sulfonyl]amino}-1-naphthoic acid;
2-{[(5-fluoro-2-methylphenyl)sulfonyl]amino}-1-naphthoic acid;
2-{[(2-methoxy-5-methylphenyl)sulfonyl]amino}-1-naphthoic acid;
2-{[(2-chloro-6-methylphenyl)sulfonyl]amino}-1-naphthoic acid;
2-{[(3,5-dichloro-2-hydroxyphenyl)sulfonyl]amino}-1-naphthoic acid;
2-({[4-chloro-3-(trifluoromethyl)phenyl]sulfonyl}amino)-1-naphthoic acid;
2-{[(2,4-dimethoxyphenyl)sulfonyl]amino}-1-naphthoic acid;
2-{[(3,4-difluorophenyl)sulfonyl]amino}-7-fluoro-1-naphthoic acid; and
2-{[(2,4-difluorophenyl)sulfonyl]amino}-7-fluoro-1-naphthoic acid.

14. The compound of claim 6 wherein R⁵ is heteroaryl.

15. The compound of claim 14 selected from the group consisting of
2-[(8-quinolinylsulfonyl)amino]-1-naphthoic acid;
2-[(2,1,3-benzothiadiazol-4-ylsulfonyl)amino]-1-naphthoic acid;
2-[(2-thienylsulfonyl)amino]-1-naphthoic acid;
2-{[(3,5-dimethyl-4-isoxazolyl)sulfonyl]amino}-1-naphthoic acid;
2-{[(5-chloro-2-thienyl)sulfonyl]amino}-1-naphthoic acid;
2-{[(5-chloro-1,3-dimethyl-1H-pyrazol-4-yl)sulfonyl]amino}-1-naphthoic acid;
2-({[2-(methoxycarbonyl)-3-thienyl]sulfonyl} amino)-1-naphthoic acid;
2-({[5-(3-isoxazolyl)-2-thienyl]sulfonyl} amino)-1-naphthoic acid;
2-{[(2,5-dichloro-3-thienyl)sulfonyl]amino}-1-naphthoic acid;
2-{[(4,5-dichloro-2-thienyl)sulfonyl]amino}-1-naphthoic acid; and
2-{[(5-bromo-6-chloro-3-pyridinyl)sulfonyl]amino}-1-naphthoic acid.

16. The compound of claim 6 wherein R⁵ is selected from the group consisting of alkyl, arylalkenyl, arylalkyl, and haloalkyl.

17. The compound of claim 16 selected from the group consisting of
2-[(butylsulfonyl)amino]-1-naphthoic acid;
2-[(benzylsulfonyl)amino]-1-naphthoic acid;
2-({[(E)-2-phenylvinyl]sulfonyl}amino)-1-naphthoic acid;
2-{[(3-chloropropyl)sulfonyl]amino}-1-naphthoic acid;
2-[(methylsulfonyl)amino]-1-naphthoic acid;
2-[(ethylsulfonyl)amino]-1-naphthoic acid; and
2-[(propylsulfonyl)amino]-1-naphthoic acid.

18. A method of inhibiting angiogenesis comprising administering to a patient in need of such treatment a therapeutically effective amount of a compound of formula (I) 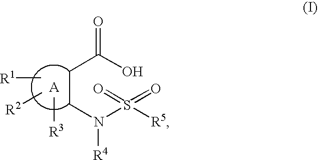
or a therapeutically acceptable salt thereof, wherein
A is a five- or six-membered aromatic or non-aromatic ring containing from zero to three atoms selected from the group consisting of nitrogen, oxygen, and sulfur; wherein the five- or six-membered ring is optionally fused to a second five-, six-, or seven-membered aromatic or non-aromatic ring containing from zero to three atoms selected from the group consisting of nitrogen, oxygen, and sulfur; R¹, R², and R³ are independently selected from the group consisting of hydrogen, alkoxy, alkoxyalkyl, alkyl, alkylsulfanyl, alkylsulfanylalkyl, amino, aminoalkyl, cycloalkyl, (cycloalkyl)alkyl, halo, haloalkoxy, haloalkyl, and hydroxyalkyl;
R⁴ is selected from the group consisting of hydrogen, alkyl, alkylsulfanylalkyl, aryl, and arylalkyl; and
R⁵ is selected from the group consisting of alkyl, amino, aminoalkyl, aryl, arylalkenyl, arylalkyl, haloalkyl, heteroaryl, heteroarylalkenyl, heteroarylalkyl, and heterocycle.

19. The method of claim 18 wherein the compound administered is a compound of formula (V) 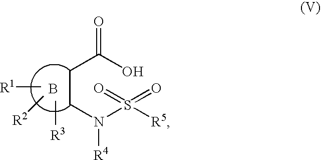
or a therapeutically acceptable salt thereof, wherein
B is a five- or six-membered carbocyclic aromatic or non-aromatic ring; and
R¹, R², R³, R⁴, and R⁵ are as defined in claim 1.

20. The method of claim 19 wherein the compound administered is a compound of formula (II) 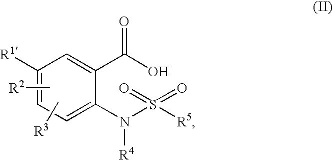
or a therapeutically acceptable salt thereof, wherein
R¹′ is selected from the group consisting of alkoxy, alkoxyalkyl, alkyl, alkylsulfanyl, alkylsulfanylalkyl, amino, aminoalkyl, cycloalkyl, (cycloalkyl)alkyl, halo, haloalkoxy, and haloalkyl; and
R², R³, R⁴, and R⁵ are as defined in claim 18.

21. The method of claim 20 wherein R⁵ is aryl.

22. The method of claim 21 wherein R⁵ is aryl wherein the aryl is unsubstituted.

23. The method of claim 22 wherein the compound of formula (VI) is selected from the group consisting of
5-ethyl-2-[(phenylsulfonyl)amino]benzoic acid;
5-isopropyl-2-[(phenylsulfonyl)amino]benzoic acid;
5-isobutyl-2-[(phenylsulfonyl)amino]benzoic acid;
2-[(phenylsulfonyl)amino]-5-propylbenzoic acid;
5-cyclopentyl-2-[(phenylsulfonyl)amino]benzoic acid;
5-cyclohexyl-2-[(phenylsulfonyl)amino]benzoic acid;
5-butyl-2-[(phenylsulfonyl)amino]benzoic acid;
5-(3-methylbutyl)-2-[(phenylsulfonyl)amino]benzoic acid;
5-(2-methylbutyl)-2-[(phenylsulfonyl)amino]benzoic acid;
5-pentyl-2-[(phenylsulfonyl)amino]benzoic acid;
5-(2-ethylbutyl)-2-[(phenylsulfonyl)amino]benzoic acid;
5-hexyl-2-[(phenylsulfonyl)amino]benzoic acid; and
5-ethyl-2-[(1−naphthylsulfonyl)amino]benzoic acid.

24. The method of claim 21 wherein R⁵ is aryl wherein the aryl is monosubstituted.

25. The method of claim 24 wherein the compound of formula (VI) is selected from the group consisting of
5-ethyl-2-{[(3-methylphenyl)sulfonyl]amino}benzoic acid;
5-ethyl-2-{[(2-fluorophenyl)sulfonyl]amino}benzoic acid;
5-ethyl-2-{[(3-fluorophenyl)sulfonyl]amino}benzoic acid;
5-ethyl-2-{[(4-fluorophenyl)sulfonyl]amino}benzoic acid;
2-{[(2-chlorophenyl)sulfonyl]amino}-5-ethylbenzoic acid;
2-{[(3-chlorophenyl)sulfonyl]amino}-5-ethylbenzoic acid;
5-ethyl-2-({[3-(trifluoromethyl)phenyl]sulfonyl}amino)benzoic acid;
2-{[(2-bromophenyl)sulfonyl]amino}-5-ethylbenzoic acid;
2-{[(3-bromophenyl)sulfonyl]amino}-5-ethylbenzoic acid;
5-ethyl-2-{[(4-methylphenyl)sulfonyl]amino}benzoic acid;
2-{[(3-cyanophenyl)sulfonyl]amino}-5-ethylbenzoic acid;
2-{[(4-cyanophenyl)sulfonyl]amino}-5-ethylbenzoic acid;
5-ethyl-2-{[(3-methoxyphenyl)sulfonyl]amino}benzoic acid;
5-ethyl-2-({[2-(methylsulfonyl)phenyl]sulfonyl}amino)benzoic acid;
5-ethyl-2-({[2-(trifluoromethoxy)phenyl]sulfonyl}amino)benzoic acid; and
2-({[5-(dimethylamino)-1-naphthyl]sulfonyl}amino)-5-ethylbenzoic acid.

26. The method of claim 21 wherein R⁵ is aryl wherein the aryl is disubstituted.

27. The method of claim 26 wherein the compound of formula (VI) is selected from the group consisting of
2-{[(2-chloro-4-fluorophenyl)sulfonyl]amino}-5-ethylbenzoic acid;
2-{[(3,4-difluorophenyl)sulfonyl]amino}-5-ethylbenzoic acid;
2-{[(2,3-dichlorophenyl)sulfonyl]amino}-5-ethylbenzoic acid;
2-{[(2,5-dichlorophenyl)sulfonyl]amino}-5-ethylbenzoic acid;
2-{[(3,5-dichlorophenyl)sulfonyl]amino}-5-ethylbenzoic acid;
2-{[(2,5-dimethylphenyl)sulfonyl]amino}-5-ethylbenzoic acid;
2-{[(3-chloro-4-fluorophenyl)sulfonyl]amino}-5-ethylbenzoic acid;
2-{[(2,5-dimethoxyphenyl)sulfonyl]amino}-5-ethylbenzoic acid;
5-ethyl-2-{[(5-fluoro-2-methylphenyl)sulfonyl]amino}benzoic acid; and
2-({[3,5-bis(trifluoromethyl)phenyl]sulfonyl}amino)-5-ethylbenzoic acid.

28. The method of claim 20 wherein R⁵ is selected from the group consisting of alkyl and heteroaryl.

29. The method of claim 28 wherein the compound of formula (VI) is
5-ethyl-2-[(8-quinolinylsulfonyl)amino]benzoic acid;
2-[(butylsulfonyl)amino]-5-ethylbenzoic acid;
5-ethyl-2-[(2-thienylsulfonyl)amino]benzoic acid;
2-{[(5-chloro-1,3-dimethyl-1H-pyrazol-4-yl)sulfonyl]amino}-5-ethylbenzoic acid; and
5-ethyl-2-({[2-(methoxycarbonyl)-3-thienyl]sulfonyl}amino)benzoic acid.

30. The method of claim 18 wherein the compound administered is a compound of formula (VI) 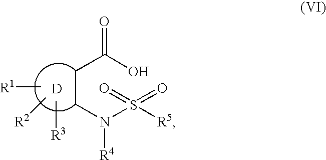
or a therapeutically acceptable salt thereof, wherein
D is a five- or six-membered aromatic-or non-aromatic ring containing from zero to three atoms selected from the group consisting of nitrogen, oxygen, and sulfur; wherein the five- or six-membered ring is fused to a second five-, six, or seven-membered aromatic or non-aromatic ring containing from zero to three atoms selected from the group consisting of nitrogen, oxygen, and sulfur; and
R¹, R², R³, R⁴, and R⁵ are as defined in claim 18.

31. The method of claim 30 wherein the compound administered is a compound of formula (III) 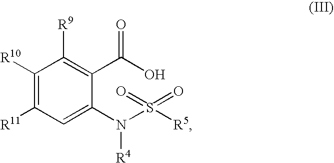
or a therapeutically acceptable salt thereof, wherein
R⁴ and R⁵ are as described in claim 18; and
R⁹ is hydrogen and R¹⁰ and R¹¹, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl; or
R¹¹ is hydrogen and R⁹ and R¹⁰, together with the carbon atoms to which they are attached, form a five-, six-, or seven-membered saturated carbocyclic ring which can be optionally substituted with one or two substituents independently selected from the group consisting of alkoxy, alkyl, amino, halo, and haloalkyl.

32. The method of claim 31 wherein R⁵ is aryl.

33. The method of claim 32 wherein R⁵ is aryl wherein the aryl is unsubstituted.

34. The method of claim 33 wherein the compound of formula (III) is selected from the group consisting of
6-[(phenylsulfonyl)amino]-5-indanecarboxylic acid;
2-[(phenylsulfonyl)amino]-1-naphthoic acid;
2-[(1−naphthylsulfonyl)amino]-1-naphthoic acid;
7-fluoro-2-[(phenylsulfonyl)amino]-1-naphthoic acid; and
2-[(phenylsulfonyl)amino]-5,6,7,8-tetrahydro-1-naphthalenecarboxylic acid.

35. The method of claim 32 wherein R⁵ is aryl wherein the aryl is monosubstituted.

36. The method of claim 35 wherein the compound of formula (III) is selected from the group consisting of
2-{[(4-chlorophenyl)sulfonyl]amino}-1-naphthoic acid;
2-{[(4-iodophenyl)sulfonyl]amino}-1-naphthoic acid;
2-{[(3-fluorophenyl)sulfonyl]amino}-1-naphthoic acid;
2-{[(4-fluorophenyl)sulfonyl]amino}-1-naphthoic acid;
2-{[(2-methylphenyl)sulfonyl]amino}-1-naphthoic acid;
2-{[(3-methylphenyl)sulfonyl]amino}-1-naphthoic acid;
2-{[(4-methylphenyl)sulfonyl]amino}-1-naphthoic acid;
2-{[(2-fluorophenyl)sulfonyl]amino}-1-naphthoic acid;
2-({[2-(trifluoromethoxy)phenyl]sulfonyl}amino)-1-naphthoic acid;
2-[({2-[(3-aminopropyl)amino]phenyl}sulfonyl)amino]-1-naphthoic acid;
2-{[(4-methoxyphenyl)sulfonyl]amino}-1-naphthoic acid;
7-fluoro-2-{[(4-fluorophenyl)sulfonyl]amino}-1-naphthoic acid;
7-fluoro-2-{[(3-fluorophenyl)sulfonyl]amino}-1-naphthoic acid; and
6-bromo-2-{[(4-fluorophenyl)sulfonyl]amino}-1-naphthoic acid.

37. The method of claim 32 wherein R⁵ is aryl wherein the aryl is disubstituted or trisubstituted.

38. The method of claim 37 wherein the compound of formula (III) is selected from the group consisting of
2-{[(3,4-difluorophenyl)sulfonyl]amino}-1-naphthoic acid;
2-{[(2-chloro-4-fluorophenyl)sulfonyl]amino}-1-naphthoic acid;
2-{[(5-fluoro-2-methylphenyl)sulfonyl]amino}-1-naphthoic acid;
2-{[(2-methoxy-5-methylphenyl)sulfonyl]amino}-1-naphthoic acid;
2-{[(2-chloro-6-methylphenyl)sulfonyl]amino}-1-naphthoic acid;
2-{[(3,5-dichloro-2-hydroxyphenyl)sulfonyl]amino}-1-naphthoic acid;
2-({[4-chloro-3-(trifluoromethyl)phenyl]sulfonyl}amino)-1-naphthoic acid;
2-{[(2,4-dimethoxyphenyl)sulfonyl]amino}-1-naphthoic acid;
2-{[(3,4-difluorophenyl)sulfonyl]amino}-7-fluoro-1-naphthoic acid; and
2-{[(2,4-difluorophenyl)sulfonyl]amino}-7-fluoro-1-naphthoic acid.

39. The method of claim 31 wherein R⁵ is heteroaryl.

40. The method of claim 39 wherein the compound of formula (III) is selected from the group consisting of
2-[(8-quinolinylsulfonyl)amino]-1-naphthoic acid;
2-[(2,1,3-benzothiadiazol-4-ylsulfonyl)amino]-1-naphthoic acid;
2-[(2-thienylsulfonyl)amino]-1-naphthoic acid;
2-{[(3,5-dimethyl-4-isoxazolyl)sulfonyl]amino}-1-naphthoic acid;
2-{[(5-chloro-2-thienyl)sulfonyl]amino}-1-naphthoic acid;
2-{[(5-chloro-1,3-dimethyl-1H-pyrazol-4-yl)sulfonyl]amino}-1-naphthoic acid;
2-({[2-(methoxycarbonyl)-3-thienyl]sulfonyl}amino)-1-naphthoic acid;
2-({[5-(3-isoxazolyl)-2-thienyl]sulfonyl}amino)-1-naphthoic acid;
2-{[(2,5-dichloro-3-thienyl)sulfonyl]amino}-1-naphthoic acid;
2-{[(4,5-dichloro-2-thienyl)sulfonyl]amino}-1-naphthoic acid; and
2-{[(5-bromo-6-chloro-3-pyridinyl)sulfonyl]amino}-1-naphthoic acid.

41. The method of claim 31 wherein R⁵ is selected from the group consisting of alkyl, arylalkenyl, arylalkyl, and haloalkyl.

42. The method of claim 41 wherein the compound of formula (III) is selected from the group consisting of
2-[(butylsulfonyl)amino]-1-naphthoic acid;
2-[(benzylsulfonyl)amino]-1-naphthoic acid;
2-({[(E)-2-phenylvinyl]sulfonyl}amino)-1-naphthoic acid;
2-{[(3-chloropropyl)sulfonyl]amino}-1-naphthoic acid;
2-[(methylsulfonyl)amino]-1-naphthoic acid;
2-[(ethylsulfonyl)amino]-1-naphthoic acid; and
2-[(propylsulfonyl)amino]-1-naphthoic acid.

43. A method of inhibiting angiogenesis comprising administering to a patient in need of such treatment a therapeutically effective amount of a compound of formula (IV)′ 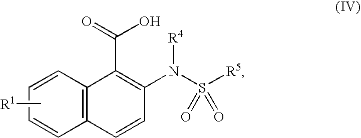
or a therapeutically acceptable salt thereof, wherein
R¹, R⁴, and R⁵ are as defined in claim 18.

44. A method of inhibiting methionine aminopeptidase-2 comprising administering to a patient in need of such treatment a therapeutically effective amount of a compound of formula (I), or a therapeutically acceptable salt thereof.

45. A method of treating cancer comprising administering to a patient in need of such treatment a therapeutically effective amount of a compound of formula (I), or a therapeutically acceptable salt thereof.

46. A method of treating cancer comprising administering to a patient in need of such treatment a therapeutically effective amount of a compound of formula (IV), or a therapeutically acceptable salt thereof.

47. A pharmaceutical composition comprising a compound of claim 1 or a therapeutically acceptable salt thereof in combination with a therapeutically acceptable carrier.

48. A pharmaceutical composition comprising a compound of claim 6 or a therapeutically acceptable salt thereof in combination with a therapeutically acceptable carrier.
